(19) United States
(12) Patent Application Publication
(10) Pub. No.: US 2003/0113797 A1
(43) Pub. Date: Jun. 19, 2003

(54) Method for generating, screening and dereplicating natural product libraries for the discovery of therapeutic agents

(75) Inventors: Qi Jia, Superior, CO (US); Mei-Feng Hong, Northglenn, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: UNIGEN PHARMACEUTICALS, INC., Broomfield, CO

(21) Appl. No.: 10/185,758

(22) Filed: Jun. 27, 2002

Related U.S. Application Data
(60) Provisional application No. 60/301,523, filed on Jun. 27, 2001.

(51) Int. Cl. G01N 33/53; G01N 33/543; A61K 35/78

(52) U.S. Cl. 435/7.1; 436/518; 424/725

(57) ABSTRACT

The present invention relates generally to a technology platform, referred to as Phytologix™ for the discovery of novel bioactive pharmaceutical, nutraceutical and cosmetic agents. Specifically, this invention includes an integrated system for the collection of medicinal plants and creation of informatic databases related to these plants. This invention also relates to an improved standardized extraction and fractionation process, which provides significant advantages over the prior art in the terms of simplicity, efficiency of the separations, the quality of the library, low cost of the process and extraordinary throughput. This invention provides details to the structure dereplication process by utilizing the technology such as HPLC/PDA/MS coupled with high throughput bioassay data and an internal pure compound library. It has been proven to be much more efficient and accurate when compared to the prior art methods. Finally, the Phytologix™ platform has been approved as a realistic and efficient process by the demonstration of the whole process of discovery and development of natural COX-2 and tyrosinase inhibitors as novel nutraceutical and cosmetic products.

### FIELD OF THE INVENTION

The present invention relates generally to a technology platform, referred to as Phytologix™, for the discovery and development of novel bioactive pharmaceutical, nutraceutical and cosmetic agents. The invention provides details on bioprospecting and informatics, parallel and preparative purification technology, online (HTP/UV/MS) and offline (HPLC/PDA/MS) dereplication, high throughput bioassay technology, a computerized database search strategy, and a conventional approach to product development in the pharmaceutical, nutraceutical and cosmetic fields.

### BACKGROUND OF THE INVENTION

Natural products have not only formed a scientific basis for the traditional use of medicinal plants, but have also played an important role in modern medicine. (Newman et al. (2000) Nat. Prod. Rep. 17:215-234). Based on a review of drugs approved between 1983 and 1994, drugs of natural origin contributed to 78% of the antibacterial drugs, 75% of platelet aggregation inhibitors, 61% of anticancer drugs, 48% of anti-hypotensive drugs, 47.6% of antiulcer drugs, and 32.5% of the anti-inflammatory drugs approved. (Cragg et al (1997) J. Nat. Prod. 60:52-60). However, analgesic, antidepressant, antihistamine, anxiolytic, cardiotonic, antifungal agents and hypnotic drugs are primarily synthetic in origin.

Natural products have been demonstrated to be highly diversified structural resources for the discovery of potential drug leads. There are over 169,000 known natural products. (The Combined Chemical Dictionary, Chapman and Hall/CRC, version 10:2 Feb. 2002). Based on the analyses of 10,495 natural products and 5757 trade drugs, it was discovered that natural products possess 1748 different ring systems, which is two times more diverse than the 807 different ring systems found in trade drugs. (Lee and Scheneider (2001) J. Com. Chem 3:284-289). Approximately 35% of the ring systems found in trade drugs are also found in natural products, however only 17% of the ring systems found in natural products have an identical counterpart in trade drugs. Natural products are not only functional as structural leads, but also have very similar architecture and pharmacophoric properties as those of trade drugs (Lee and Scheneider, (2001) J. Corn. Chem 3:284-289; Bemis and Murcko (1996) J. Med. Chem. 39:2887-2893). In a comparison of 10,495 natural products with 5757 trade drugs, it has been found that the average calculated molecular weight of natural products is almost identical to that of trade drugs (356 vs. 360); and the average log p values are slightly higher for the natural products (2.9) than for trade drugs (2.5). Natural products have fewer hydrogen donors per molecule and fewer nitrogens per molecule than trade drugs; have a much higher number of bridgehead atoms than trade drugs and synthetic drugs; and have many more chiral centers per molecule (Henkel et al. (1999) Angew. Chem. Int. Ed. 38:643-647). However, both natural products and trade drugs have a similar average number of oxygens per molecule and the same percentage of compounds with at least two “rule-of-5” violations. (Lipinski et al. (1997) Adv. Drug Delivery Rev. 23:3-25).

With the advancement of new technology, such as combinatorial syntheses, computational drug design and super high throughput screening, there has been an increasing interest in the design of small molecule libraries using natural products as templates. (Hall et al. (2001) J. Combinatorial Chem 3(2):125-150; Wang and Ramnarayan (1999) J. Comb. Chem. 1:524-533). Combinatorial libraries can be generated in solution, however, most of the libraries generated to date rely on solid-phase synthetic techniques, including solid-phase extractions, which are used predominantly in the purification of the targeted synthetic compounds. (Desai et al. (1994) Drug Devel. Res. 33:174-188). Unfortunately, there are significant limitations in the synthetic approach to generating libraries from complex natural product templates, particularly with compounds containing multiple-rings and multiple chiral center skeletons. An obvious limitation, for a semi-synthetic approach is that certain skeletal modifications and crucial functional group positions can not be diversified. To date, all published compound libraries have been generated using collections of starting materials and a certain reaction or reaction sequence that must be optimized under specific conditions. (Weber (2000) Current Opinion in Chem Biol. 4:295-302). Additionally, to develop a synthetic library from a known natural product lead, requires a significant amount of information regarding the relationship between structure and activity to define the potential sites on the natural product template that could be modified. Thus, the general approach to combinatorial synthetic chemistry involves the identification of a specific type of natural product based upon available pharmacological profiles and the dissection of structures into scaffolds or templates.

The design of focused natural product libraries has its roots in combinatorial synthesis and computational chemistry. (Wessjohann (2000) Current Opinion in Chem Biol. 4:303-309; Kolb (1998) Prog. Drug Res. 51:185-217). Efforts have been made to design specific types of libraries that target specific types of compounds (Stahura et al. (2000) J. Med. Model 6:550-562), that focus on specific therapeutic targets or that include bioavailability as a criteria (Shu (1996) J. Nat Prod. 61:1053-1071). Many different types of natural product templates have been developed and natural product libraries have been successfully generated, including alkaloid like libraries, from compounds such as, benzylamines (Green (1995), J. Org. Chem. 60:4287-4290), quinazolines (Wang and Ganesan (2000) J. Comb. Chem. 2:186-194), indoly diketopiperazines (Loevezijin et al. (1998) Tetrahedron Lett. 39:4737-4740), mappicine analogues (Josien and Curran (1997) Tetrahedron 53:8881-8886), yohimbine analogues (Ni et al. (1996) J. Med. Chem 39:1601, Atuegbu et al. (1996) 4:1097-1106) and oligoheterocycles (Boger et al. (2000) J. Am. Chem. Soc. 122:6382-6394); and flavonoid like libraries, from compounds such as, flavone analogues (Marder et al. (1998) Biochem. Biophys. Res. Commun 249:481-485), and benzopyrans (Nicolaou et al. (2000) J. Am. Chem. Soc. 122:9939-9976, Mason et al. (1999) J. Med. Chem. 42:3251-3264).

Synthetic libraries generally contain purified single compounds in a quantity of 1-2 mg, with a purity of approximately 70-80% based on HPLC. Due to the co-existence of other chemical components resulting from the synthetic processes, the biological screening assays may be significantly impacted by false positives, false negatives and other complications. Designing a combinatorial library demands careful optimization of reaction selectivity and efficiency to avoid low yield, difficulty of purification and loss of chiral centers. It has been demonstrated that a specific, desirable biological property of a natural product can be improved even with rather small libraries integrating simple functional group modifications. (Hall et al. (2001) J Combinatorial Chem. 3(2):125-150). To date, there are few reports of the use of combinatorial libraries in the agriculture and food industries (Wang and Rebintson (1999) in Chemicals Via Higher Plant Bioengineering, Shahidi ed., Kluwer Academic/Plenum Publ. Pp 91-105). Additionally, there are no reports on the application of such libraries in the dietary supplements and cosmetics industries.

Grabley et al. have published an extensive review on the discovery of drugs from natural product-based libraries. (Grabley et al. (2000) Ernst Schering Res. Found Workshop 32:217-252). The screening of natural products typically begins with crude extracts. Specifically, the biomass of the plant is extracted multiple times with multiple solvents, which are typically chosen based upon their polarity. Unfortunately, these crude extracts contain large numbers of compounds, which are present in low concentrations. This typically results in the identification of biological activity resulting from the major components only. Compounds with potent activity, but present in concentrations below the detection limits may be missed altogether. Additionally, this may lead to false positive results, due to synergistic effects from similar weakly active components, or to non-specific interference from common components.

To date, there have been few reports of methods to generate natural product libraries directly from natural sources that are suitable for high throughput screenings and product discovery. One such method, was recently reported by Gary et al. (2000) WO 00133193. The method of Gary et al. comprises the steps of (a) thoroughly extracting a biological source material with alcohol/water or hexane followed by alcohol/water; (b) removing bioassay interferences from the solvent extracts by elution of the extracts through a polyamide column, (c) subjecting the eluent from the polyamide column to a solid phase extraction process with step gradients to collect limited fractions (typically 4); (d) further purifying each of these fractions by HPLC to generate the compound library based on detecting the bioactive compounds; and (e) collecting purified compounds with standardized concentrations generated by an automated system. This methodology has several drawbacks. First, the use of mixtures of alcohol/water as an extraction solvent will not extract all potential biologically active components from the biomass. A good example is polysaccharides, which will not dissolve in alcohol/water and therefore, would not be extracted from the plant biomass. However, polysaccharides are a very important class of natural products having known immune regulatory and anti-tumor effects and have been used in the pharmaceutical, nutraceutical and cosmetic industries. Second, polyphenol and tannins are biologically active ingredients (Kolodziej et al. (2001) Planta Med. 67:825-832; Abe et al. (2001) J. Nat. Prod. 64:1010-1014) that contribute to the efficacies of many popular herbal products, such as EGCG and other catechin and phenolic compounds from green teas, (No et al. (1999) Life Sci. 65:PL241-246), grape seeds and grape skins (Cantos et al. (2001) J Agric Food Chem. 49:5052-8). Removal of these components from plant extracts using the method described by Gary et al. will result in a significant loss of bioactive components that have been demonstrated to be efficacious and valuable in the prevention and treatment of diseases. Third, the process disclosed by Gary et al. is a time consuming and expensive process, requiring the use of multiple solid phase extractions and column chromatography to generate the final compound library. Finally, the inventors emphasize the known concentration and structure information for each well before understanding the potential biological profile or value. Such efforts will also be very expensive and time consuming to analyze, sort and store.

A collaborative project, designed to generate a non-redundant pure compound library with a collection of 6,700 chemical entities in a quantity of ≧5 mg and a purity of ≧80%, was reported by Bindseil et al. (Drug Discovery Today 6: 840-847 (2001)). The biomaterials consisted of 679 species of plants, 2665 bacterial strains and 1425 fungal strains. The biomaterials were pre-screened before extraction for non-ubiquitous secondary metabolisms using HPLC/ELSD/DAD and LC/MS. The isolation was then carried out via flash column chromatography and the structural information was collected and the full structure of 400 randomly selected compounds was determined. The structure dereplication procedures included a search referenced retention times and molecular weights based on LC/MS data and comparison with a commercial database (Dictionary of Natural Products). 2D-NMR and other techniques were utilized to further define substructures and provide full structural elucidation. The pure compound library generated from the above method has been screened against nine different targets and has been shown to be superior to synthetic libraries with regard to response rates and confirmation rates.

Stewart et al. have reported on the efforts at Molecular Nature Ltd to generate a pure natural product library. (Stewart et al. (2000) Saponins, in Food, Feedstuffs and Medicinal Plants, Oleszek and Marston (eds.) pp. 73-77). Compounds for this library were isolated utilizing parallel normal phase column chromatography, followed by C-18 and/or ion exchange chromatography. To be accepted into the library the compounds must be >90% pure with structural verification by a combination of HPLC, NMR, MS and GC/MS. A method to make a secondary metabolite library from a microbial culture broth was reported by Schmid et al. (J. Biomol. Screening 4:15-25 (1999)). The library was generated using a novel automated process based on multistep fractionation of a supernatant from broth through an Amberlite XAD-16 column, followed by chromatographic column fractionations with a styrene-divinylbenzene resin, reverse phase C-8 and C-18 and other types of solid phase extractions (SPE). This effort led to higher purity compounds in each fraction based on an automatic procedure with limited manual intervention. However, this method has several limitations. For example, SPE uses step-gradients that lead to limited fraction numbers in large volumes, and it is not a suitable method to collect fractions in a 96-well format. Finally, Dr. Kingston has disclosed the generation of a natural combinatorial library for anticancer drug discovery. (Kingston (2001) Abs. Papers Amer. Chem. Soc. 221:ORGN 199; (1997) Abs. Papers Amer. Chem. Soc. 214: AGRO124).

Technological development in genomics, enzymology and bioengineering has resulted in a method for generating natural products utilizing combinatorial biosynthesis. (Khosla (2000) J. Org. Chem 65: 8127-8133, Hutchinson (1998) Current Opinion Micorb. 1: 319-329). For example, multiple genetic modifications of the erythromycin polyketide synthase have produced a novel unnatural natural product library. (McDaniel (1999) Proc. Natl. Acad. Sci. USA 96:1846-1851). Combinatorial biosynthetic libraries have been constructed by cloning large fragments of DNA isolated from soil into a Streptomycete host (Wang et al. (2000) Org. Lett. 2:2401-2404), and through the glycosyltransferase catalyzed transformation (Thorson et al. (2001) Abst. Papers Amer. Chem. Soc. 221:Carb 19).

Recent developments in high throughput purification, LC/PDA, LC/MS/MS and LC/NMR for online structure dereplication and creation of informatic databases have fundamentally changed the way in which bioactive natural products are studied. The primary goal of dereplication is to identify known compounds from active extracts or fractions to avoid unnecessarily isolating these known compounds. The selection of an adequate structure database to evaluate information collected is critical to the dereplication process. (Corley and Durley (1994) J. Nat Prod. 57:1484-1490). Chemical Abstracts Service's Registration File, which includes CA, NAPROLERT, REGISTRY, BEILSTEIN, MEDLINE etc. sub-databases and the Dictionary of Natural Products, which includes the Bioactive Natural Product Database and DEREP databases are two of the most comprehensive databases

Dereplication of active crude extracts using HPLC/UV/MS, coupled with biological activity data obtained on subfractions was reported by Cordell and Shin. (Pure Appl. Chem. 71:1089-1094(1999)). In this study, active plant extracts were analyzed using a HPLC C-18 column eluting with an acetonitrile/water gradient in 30 minutes with single wavelength UV detection and ESI-MS in a positive and negative dual mode. The UV absorption properties, molecular weight and ion fragmentation information from ion chromatograms (ELC) of extracts were analyzed using NAPRALERT and the Dictionary of Natural Products. LC-ESI-MS technology was also utilized for quantitatively differentiating crude natural extracts as described by Julian et al. (1998), Anal. Chem. 70:3249-3254. Briefly, ethanol/water extracts from fungal cultures were separated on a dual-column HPLC system with C-18 columns in 25 minutes using an acetonitrile/water/ammonium acetate gradient. A Similarity Index was based on the HPLC retention time and mass to charge ratio from the positive ion mode of an ESI-MS instrument. This methodology, however, was restricted to a qualitative result with limited structural information and limited types of compounds that give a reasonable molecular ion peak in the positive mode. As demonstrated by Wolfender's report (Wolfender et al. (1995) J. Mass Spectr. Repid Commun. In Mass Spectr. S35-S46), there is no single ionization interface allowing the optimum ionization of all the secondary metabolites within a single crude plant extract. Different ionization techniques, such as ES, APCI, TSP or CF-FAB are required in conjunction with LC/DAD and MS/MS. To generate a searchable library of MS/MS fragmentation spectra with reliable reproducibility it is very helpful to expand the structural information collected from mass spectrometry. (Baumann et al. (2000) Rapid Comm. In Mass Spectr. 14:349-356).

Bradshaw et al. have disclosed a rapid and facile method for the dereplication of a purified natural product library. (Bradshaw et al. (2001) J. Nat. Prod. 64:1541-1544). The method involves searching a text file that links each structure with its molecular weight from LC/MS and an exact count of the number of methyl, methylene and methane groups derived from NMR data. The search uses customized software with chemical structure information in a specific format—SMILES which has been converted from commercial databases.

The chemical structure of natural products can be identified quickly, with a limited amount of materials by utilizing NMR equipment containing cryo probes (Russell et aL (2000) J. Nat Prod. 63:1047-1049). More predictable chemical shifts, coupled with a reasonable amount of published and internal NMR data (Smith et al (2001) J. Chem. Inf. Comput. Sci. 41:1463-1469) will significantly improve the time and accuracy of the structure elucidation process (Patchkovskii and Thiel (1999) J. Computational Chem. 20:1220-1245; Grzonka and Davies (1998) J. Chem. Inf. Comput. Sci. 38:1096-1101; Schütz et al. (1997) Fresenius J. Anal. Chem. 359:33-41). Additionally, the direct coupling of HPLC with NMR and mass spectrometry (MS) provides much more structural information and significantly enhances the quality of the conclusions in the dereplication process (Lindon et al. (2000) J. Chromatogr. B 748: 233-258).

The development of high throughput screening technology began in the mid 1980's. Robotic operation coupled with laboratory information management systems, in combination with miniaturized signal reading systems, enable the throughput screening of literally millions of samples per assay per annum (Lin (1995) J. Food &Drug Anal. 3:233-242). Natural product libraries have been screened against a variety of biological (Virador et al. (1999) Analytical Biochemistry 270:207-219), biochemical (Noreen et al. (1998) J. Nat. Prod. 61:2-7) and genomic targets (Ghai (1999) U.S. Pat. No. 5,955,269). Display cloning technology has been developed for functional identification of natural product receptors using cDNA-phage display (Sche et al. (1999) Chem Biol. 6:707-716).

Inhibition of the enzyme cyclooxygenase (COX) is the mechanism of action attributed to most nonsteroidal anti-inflammatory drugs (NSAIDS). There are two distinct isoforms of the COX enzyme (COX-1 and COX-2) that share approximately 60% sequence homology, but differ in expression profiles and function. COX-1 is a constitutive form of the enzyme that has been linked to the production of physiologically important prostaglandins, which help regulate normal physiological functions, such as platelet aggregation, protection of cell function in the stomach and maintenance of normal kidney function. (Dannhardt and Kiefer (2001) Eur. J. Med. Chem. 36:109-26). The second isoform, COX-2, is a form of the enzyme that is inducible by pro-inflammatory cytokines, such as interleukin-1β(IL-1β) and other growth factors. (Herschmann (1994) Cancer Metastasis Rev. 134:241-56; Xie et al. (1992) Drugs Dev. Res. 25:249-65). This isoform catalyzes the production of prostaglandin E2 (PGE2) from arachidonic acid (AA). Inhibition of COX-2 is responsible for the anti-inflammatory activities of conventional NSAIDs.

Although, rheumatoid arthritis is largely an auto-immune disease and osteoarthritis is caused by the degradation of cartilage in joints, reducing the inflammation associated with each provides a significant increase in the quality of life for those suffering from these diseases. (Wienberg (2001) Immunol. Res. 22:319-41; Wollhiem (2000) Curr. Opin. Rheum. 13:193-201). In addition to rheumatoid arthritis, inflammation is a component of rheumatic diseases in general. Therefore, the use of COX inhibitors has been expanded to include diseases, such as systemic lupus erythromatosus (SLE) (Goebel et al. (1999) Chem. Res. Tox. 12:488-500; Patrono et al. (1985) J. Clin. Invest. 76:1011-1018), as well as, rheumatic skin conditions, such as scleroderma. COX inhibitors are also used for the relief of inflammatory skin conditions that are not of rheumatic origin, such as psoriasis, in which reducing the inflammation resulting from the over production of prostaglandins could provide a direct benefit. (Fogh et al. (1993) Acta Derm Venerologica 73:191-3). Simply stated, COX inhibitors are useful for the treatment of symptoms of chronic inflammatory diseases, as well as, the occasional ache and pain resulting from transient inflammation.

### SUMMARY OF THE INVENTION

The present invention relates generally to a technology platform, referred to as Phytologix™, for the discovery and development of novel bioactive pharmaceutical, nutraceutical and cosmetic agents. The invention provides details on bioprospecting and informatics, parallel and preparative purification technology, online (HTP/UV/MS) and offline (HPLC/PDAIMS) dereplication, high throughput bioassay technology, a computerized database search strategy, and a conventional approach to product development in the pharmaceutical, nutraceutical and cosmetic fields. The method for discovering and developing novel therapeutic pharmaceutical, nutraceutical and cosmetic agents is comprised of the steps of: (a) identifying and collecting a biological sample; (b) extracting the sample using a two solvent system extraction procedure; (c) separating the extracts using two separate high throughput (HTP) fractionating methods and simultaneously determining the activity of each HTP fraction; (d) dereplicating the active fractions to identify the compounds present; and (e) generating an indication, pharmacological and safety profile for each novel compound from step (d). The sample can be selected from any natural source including, but not limited to materials of botanic, microbial, fungal, mineral, marine, animal and human origin. In a preferred embodiment the sample is a plant. Additionally, in a preferred embodiment the sample is pre-selected based upon documented traditional use or known medicinal property.

A collection form is prepared for each sample collected. The collection form contains specific information about the sample including, but not limited to Latin name, distribution, collection location, therapeutic information, traditional preparations, botanical identification and published references. This information is then transferred to a database. Specific macros and queries are designed to assess this information and data stored.

After the sample is collected, at least two specimen vouchers are prepared for each sample, wherein said specimen vouchers are comprised of dried, and/or preserved naturally and/or chemically the whole body of the sample including the full reproduction organs and wherein a taxonomy form is attached to each voucher specimen for purposes of identification. The specimen vouchers are critical and unique to guarantee the integrity and authenticity of the sample during the research stage of the process and to ensure the potential of successful recollection and production during the production stage of the process.

The second and third steps of the Phytologix™ process include multiple standardized extraction and fractionation protocols that enable the generation of diversified crude extracts and a fraction library using a high throughput procedure. The solvent extraction procedure of step (b) comprises the steps of: (a) grinding an appropriate amount of sample; (b) extracting the ground sample with a combination of two organic solvents, wherein said combination is comprised of a solvent of low polarity and a solvent of high polarity; (c) drying the sample after organic extraction; (d) extracting the dried sample with an aqueous solvent; and (e) evaporating the solvent from both extractions and isolating the extract. The amount of sample extracted is typically between 1 gram to 1000 grams.

The low polarity used in the organic extraction step is selected from the group consisting of an alkane having 6-10 carbons, a halogenated alkane having 1-4 carbon atoms, wherein each carbon atom has 1-4 halogen atoms, an ester having the formula R′COOR″, wherein R′ is selected from an alkyl group having between 1-6 carbons and R″ is selected from an alkyl group having between 1-8 carbons and a ketone having between 3-12 carbons. The low polarity solvent is selected from the group consisting of methylene chloride, ethyl acetate and chloroform. The high polarity solvent is selected from the group consisting of DMSO, THF and an alcohol, wherein said alcohol has one to eight carbons. In a preferred embodiment, the alcohol is selected from the group consisting of methanol, ethanol, propanols and butanols. The aqueous solvent is selected from the group including, but not limited to, water, acidic water, basic water, or an aqueous buffer, wherein the pH is adjusted between one to fourteen. The extraction can be carried out using any method known in the art for extraction including, but not limited to, shaking, sonication, refluxing, stirring, and pressurized mixing, and filtering.

The extracts obtained from the extraction process are prepared for bioassay by (a) weighing and dissolving the organic extract into a solvent; (b) weighing and dissolving aqueous extract in a solvent; and (c) transferring each extract solution into individual cells of a sample master plate. The solvent for dissolving the organic and aqueous extracts are independently selected from the group of solvents including, but not limited to, DMSO, DMF, THF, ketones having three to ten carbons and alcohols having one to five carbons.

The extracts obtained are then separately fractionated using a parallel chromatography system or a high throughput purification (HTP) system by a method comprising the steps of (a) separating the organic extract with a normal phase pre-packed column; (b) separating the aqueous extract with a reverse phase pre-packed column; (c) detecting eluent with detector(s); (d) collecting fractions; and (e) evaporating the solvent. The chromatography/HTP is carried out at ambient, low, medium or high solvent pressure and at ambient, or a temperature from 20 to 80° C. The normal phase column is packed with a resin selected from the group consisting of silica gel, alumina, and amino propyl, cyano propyl, diol florisil or polyamide, ion exchange resins. The reverse phase column is packed with a resin selected from the group consisting of C-2, C-4, C-8, C-18, LH-20, XAD-4, XAD-16, and polystyrene-divinyl benzene based resins. The particle size of the resin in each chromatography column is from 10 to 200 μm and the chromatography column is packed with 1 to 500 grams of resin depending upon the amount of sample and difficulty of separation.

The normal phase chromatography column is eluted with a combination of three organic solvents selected from an alkane having six to ten carbons, an organic ester, having the formula R¹COOR², wherein R¹ is selected from an alkyl group having between one to five carbon and R² is selected from an alkyl group having between one to six carbons, and an alcohol, having the formula R³OH, wherein R³ is an alkyl group having between one to six carbons. The reverse phase chromatography column is eluted with a combination of two solvents: deionized (DI) water and a solvent selected from the group consisting of an alcohol with one to four carbons, acetonitrile, THF, or a ketone having three to twelve carbons.

The detector may be any detector used in the art for such purposes including, but not limited to an ultraviolet (UV)/visual light detector, a Mass Spectrometer (MS) detector, a Nuclear Magnetic Resonance (NMR) detector, a reflex index (RI) detector or a light scattering detector (LSD). The ultraviolet (UV)/visual light detector may be comprised of single or dual channels with single, continuing or broadband wavelength from 100-1000 nm. The MS detector may be comprised of an electronic spray ionization or sonic spray ionization chamber; ion trap or single or triple quadruple mass detection with positive or negative mode. The NMR detector may be comprised of a proton or carbon probe.

After HTP fractionation each of the fractions is tested for bioactivity. In a preferred embodiment the bioassay is performed simultaneously with the HTP fractionation. The method for preparing the individual fractions for bioassay comprises the steps of: (a) dissolving the fractions from organic extract into a solvent; (b) dissolving the fractions from aqueous extract into a solvent; and (c) transferring the fraction solution into a sample plate. The solvent for dissolving the fractions derived from the organic extract and the aqueous extract is independently selected from the group including, but not limited to DMSO, DMF, THF, a ketone containing three to ten carbons, an alcohol containing one to five carbons and a combination of two to three of solvents. Each fraction is then assayed using standard biochemical (enzymatic), functional or biological models as the primary screening method to identify extracts and compounds with a particular activity.

Once active botanical extracts and/or fractions, and/or compounds are identified as having a mechanism of action and/or a specific therapeutic value, chemical composition profiling and active component standardization will be carried out. Thus, once identified, the active fractions are subjected to a dereplicating process which comprises the steps of: (a) collecting activity data related to the sample; (b) collecting physical property, spectroscopic and structural data related to the sample; (c) analyzing the collected data; (d) searching commercial databases for the properties of the sample; and (e) reaching a conclusion regarding the composition of the active fractions.

The activity of the samples is measured using standard means including, but not limited to enzyme inhibition, receptor binding, gene expression, cell function regulation, protein production, animal function regulation and animal disease model manipulation and other measurements of biological function. The activity data can be collected from extracts, fractions of extracts, purified compounds, semi-synthetic and synthetic compounds. Physical property data collected in the dereplication process includes, but is not limited to, retention time from a chromatogram based on absorption or changes of UV/VIS, refractive index, laser light scattering pattern, solvent elution volume, mass weight, pH, solubility and log P.

The spectroscopic information collected includes, but is not limited to, UV/VIS spectrum, mass spectrum including molecular ion and fragmentation ions, NMR spectrum and light scattering spectrum. Structural information is obtained from data such as mass fragmentation pattern and mass spectrum of daughter/grand daughter ions; chemical shifts of protons, carbons, phosphorous, and other elements from one and two dimensional nuclear magnetic resonance spectroscopic data; infrared spectrum and UV absorption spectrum. The data collection process can be an online method by splitting a portion of eluent into a designated detector(s) and/or an offline process by analyzing individual samples after collected from the HTP separation. The data collected is then analyzed using various databases. Commercial databases that can be used include, but are not limited to the Dictionary of Natural Products, Chemical Abstracts Service's Registration File, NAPROLERT, MEDLINE, NERAC, DEREP and the Bioactive Natural Product Database.

This process results in the dereplication of the composition in each fraction. If the composition is determined to be novel, further studies are carried out to generate an indication, pharmacological and safety profile for each novel natural product. If these results are positive the compound is then developed into a commercially viable product.

It is to be understood that both the foregoing general description and the following detailed description are exemplary and explanatory only and are not restrictive of the invention as claimed.

### BRIEF DESCRIPTION OF THE FIGURES

FIG. 1 illustrates a representative collection form submitted by medicinal plant collectors. The illustrative collection form covers information regarding plant origin, botanical identification, geological distribution, ethno indications, chemical components and references.

FIG. 2 illustrates the tables and relationships of those tables in a database that covers all information about the plants, research data and publication references.

FIG. 3 depicts the macros designed to draw information from the tables in the database in order to generate final reports based on specific queries.

FIG. 4 illustrates a representative plant information overview on Polygonum viviparum, which includes botanical information, plant weights, extract weights and ethno indications.

FIG. 5 depicts the HPLC/UV chromatograms of the organic extract (FIG. 5A), aqueous extract (FIG. 5B) and methanol extract (FIG. 5C) from the flowers of Daphne genkwa (P0490). There were no specific peaks present in the methanol extract that were not also present in either the organic extract or the aqueous extract.

FIG. 6 depicts the HPLCIMS total ion chromatograms (TIC) of the organic extract (FIG. 6A), aqueous extract (FIG. 6B) and methanol extract (FIG. 6C) from the flowers of Daphne genkwa (P0490). There were no specific peaks present in the methanol extract which were not present in either the organic extract or the aqueous extract.

FIG. 7 illustrates the separation efficiency of high throughput purification system on an organic extract from the roots of Pulsatilla chinensis. Every other HTP fractions were spotted and developed on a silica gel TLC plate and developed with 60% EtOAc in Hexane. The TLC plate was spread with coloration agent anialdehyde in sulfuric acid.

FIG. 8 depicts the weight distribution of each HTP fraction in the 96-deep well plate collected from fractionation of the organic extract from the roots of Pulsatilla chinensis.

FIGS. 9A-9L illustrate the reproducibility of the high throughput purification system disclosed herein. Specifically, they depict 12 HTP/UV chromatograms from twelve reverse phase C-18 column fractionations of the same aqueous extract isolated from the whole plant of Ainsliaea henryi.

FIG. 10 illustrates the positive hit rate resulting from the screening of 1230 plant extracts for COX inhibitory activity. The positive hit rate was 1.2% positive for organic extracts and 0.6% for aqueous extracts. This screening resulted in the identification of 22 active plant extracts.

FIG. 11 illustrates the tyrosinase inhibition distribution pattern of 396 organic extracts from various species of plants. A total of 36 plant extracts showed >60% inhibition of tyrosinase activity with 9.1% positive hit rate.

FIG. 12 depicts the HTP/UV chromatogram of reverse phase fractionation of aqueous extract from the leaves of Camellia sinensis (P0605).

FIG. 13 depicts graphically the inhibition of COX-1 (▪) and COX-2 (♦) by various HTP fractions from the aqueous extract of the leaves of Camellia sinensis (P0605).

FIG. 14 depicts the online PDA/MS base ion chromatogram (BIC) of bioactive HTP fraction D3, derived from an aqueous extract of the leaves of Camellia sinensis (P0605).

FIG. 15 illustrates the HPLC/PDA chromatogram (FIG. 15A) and HPLC/MS total ion chromatogram (TIC) (FIG. 15B) from the off-line analysis of HTP bioactive fraction D3, derived from an aqueous extract of the leaves of Camellia sinensis (P0605).

FIG. 16A depicts the identical mass spectra of HTP bioactive fraction D3 based on the data collection from on-line HTP/MS and off-line HPLC/MS. FIG. 16B illustrates the results of the dereplication procedure described in Example 11. As can be seen in FIG. 16B, fraction D3 contained a single known compound—Epigallocatechin gallate—whose structure is set forth in the figure.

FIG. 17 depicts the results of dereplication of all 16 bioactive HTP fractions from the aqueous extract of Camellia sinensis (P0605). There were 10 compounds present in the 24 HTP fractions, all of which had known structures, as set forth in FIG. 17.

FIG. 18 illustrates the melanin production inhibitory activity versus cell toxicity of HTP fractions from the organic extract of the whole plant of Mallotus repandus (P0368). The multiple peaks exhibiting melanin production inhibitory activity, indicates that a number of active components exist in the crude extract. The peak located from fraction C10 to D12 is a false peak, resulting from cytotoxicity.

FIG. 19 illustrates the results of the dereplication of the active peak identified from the melanin inhibition assay of the HTP fractions of the organic extract derived from Mallotus repandus (whole plant) (P0368). FIGS. 19A-F depict the total ion chromatograms of active fractions D2 to D7 collected from off-line LC/MS. The peak located at a retention time of 16.33 minutes, which showed up in fractions D3-D6, matches exactly the peak of tyrosinase inhibition. FIG. 19G depicts the mass spectrum of this peak (Rt=16.33 min.) from fraction D4. Dereplication resulted in the identification of the known polyphenol Pterocaryanin B (FIG. 19H).

FIG. 20 depicts graphically a profile of the inhibition of COX-1 and COX-2 by the isolated free-B-ring flavonoid, Baicalein, from the roots of Scutellaria baicalensis (P0483). The compound was examined for its inhibition of the peroxidase activity of recombinant ovine COX-1 (♦) or ovine COX-2 (□). The data is presented as percent inhibition of assays without inhibitor. The IC₅₀ for COX-1 was 0.18 μg/mL/unit of enzyme while the IC₅₀ for COX-2 was 0.48 μg/mL/unit.

FIG. 21 illustrates the inhibition of arachidonic acid induced inflammation by a standardized Free-B-Ring Flavonoid extract isolated from the roots of Scutellaria baicalensis. The in vivo efficacy was evaluated based on the ability to inhibit swelling induced by direct application of arachidonic acid. The average differences in swelling between the treated ears and control ears are represented in FIG. 21A. FIG. 21B illustrates the percent inhibition of each group in comparison to the arachidonic acid treated control.

FIG. 22 depicts a sale sheet for the dietary supplement Univestin™, which was discovered and developed using the Phytologix™ technology platform of this invention.

FIG. 23 illustrates the certificate of analysis (COA) for one representative batch of Univestin™ as a commercial product sold in nutraceutical and cosmetic markets.

FIG. 24 depicts the Phytologix™ discovery process schematically. From the analysis of plant collection libraries and market requirements, high throughput screening models were developed to assay the prioritized plant extracts. After identification of the biological activity, the pharmacological and safety profiles were generated based on a standardized extract/enriched fractions/pure compound. The output of this process is a product candidate.

FIG. 25 illustrates the Phytologix™ development process schematically. The product candidate, information search and product development leads to the identification of plant sources for production usage, to make recommendations on intellectual property position and market advantage. Manufacturing process development and production of pilot scale prototype product would be followed with confirmation of efficacy and safety profiles. The completion of the Phytologix™ process would be marked with successful clinical trials and final product launch.

FIG. 26 illustrates a critical task checklist that may be utilized in the Phytologix™ process to keep track of critical activities and data generation.

FIG. 27 demonstrates the time, cost estimation and decision making process in the Phytologix™ platform. It shows the requirement of full time employees, and the time and cost involved for each stage of discovery and development. It gives the project manager an opportunity to evaluate the progress of the project at each critical decision making point.

### DETAILED DESCRIPTION OF THE INVENTION

Various terms are used herein to refer to aspects of the present invention. To aid in the clarification of the description of the components of this invention, the following definitions are provided.

As used herein a “sample” refers to a biological or natural material selected from the group consisting of materials of botanic, microbial, fungal, mineral, marine, animal or human origin. In a preferred embodiment of the invention the sample is a medicinal plant. The terms “specimen” and “biomass” are used interchangeably with the term sample. In a preferred embodiment the sample is a plant.

“Nutraceutical” as used herein refers to a composition of matter targeted to an industry or market that has been defined by the “Dietary Supplement Health and Education Act of 1994 (DSHEA)” and targets humans, as well as, other animals.

“Cosmetic” as used herein refers to a composition of matter directed to an industry or market that targets prevention, treatment and maintenance of normal function, appearance and integrity of the skin, hair, figure and other physical appearance of humans, as well as, other animals.

“Natural product” refers to an element, compound, secondary metabolite or structural component that exists in natural resources. A natural product could be a single compound or a mixture of multiple compounds.

“Natural material” refers to the original material obtained directly from natural resources. It may be either the whole plant or part of a plant, an animal, a marine material, a microbial fermentation batch, a soil sample, a piece of mineral material, etc.

“Extraction” refers to a process used to isolate natural products from a natural material with a solvent, supercritical fluid, by a distillation, pressing, or sublimation processes. The output of the extraction process is called an “extract.” Any known method of extraction can be used with the method of this invention.

“Fractionation” refers to a process to separate an extract into multiple parts or fractions that contain a single or a mixture of natural products.

“Dereplication” refers to a process to analyze without isolation a natural product, a fraction or an extract for physical, spectroscopic and structural information; to compare the information with internal and commercial databases; and to reach a conclusion on the existence of novel and/or known compounds. Dereplication is used to determine how to direct further investigations.

An “active agent” and/or “biologically active agent” and/or “bioactive agent” refers to a biological function of a natural product. Examples of biological activity include, but are not limited to enzyme inhibition, receptor binding, impact on gene expression, cell function regulation, change of protein production, animal function regulation and animal disease model manipulation, as well as effects on other measurements of biological output.

A “relational database” refers to a computerized data management system that stores and retrieves data, processes and presents information and automates repetitive tasks.

A “macro” refers to computer software codes, such as “Visual Basic” or “VBA language” that enable the database to perform a designated action for automating a particular task or series of tasks.

A “query” refers to a question or inquiry posted to the relational database regarding the information stored in tables within the database. A specifically designed query can choose data from specific tables, sort and filter the data, perform calculations, create tables, forms, graphs and reports.

“Concentration” refers to amount of an extract, a fraction, or a natural product in a given volume of solvent. The extract plates are prepared with similar concentrations of extract and the fraction plates with variable concentrations in each cell that reflect the normal distribution of a natural product based on its physical properties and behaviors on a column. The concentration peak, which is revealed in the dereplication process and matched with biological profiles, is critical information for the identification of bioactive components. The concentration of a natural product must be adjusted based on the sensitivity and properties of the bioassay or screening models.

“Bioassay” or “biological screening” refers to an in vitro and/or an in vivo biological, biochemical or genomic function model(s) and a testing process that measures the effects of a natural product.

“High throughput purification” or “parallel chromatography” refers to a method designed to perform one set of multiple column separations, while simultaneously washing and equilibrating another set of columns. The process is performed on an instrument that is controlled by computer software.

A “pre-packed column” refers to a column that has been prepared based on a standardized packing protocol with the same type, quantity, particle size of resin and into the same size and diameter of column. It may be packed internally or purchased as a commercial product.

“Chromatogram” refers to an illustration of a chromatographic eluent based on the UV/VIS absorption, ionization intensity, nuclear magnetic resonance signals, light scattering capability, reflect index and other physical properties of the components of the eluent that are detected and elicited by passing the eluent through a specific detector.

A “novel compound” refers to a natural product with unknown chemical structure and composition; and/or known chemical structure, having a new biological activity/function.

A “known compound” refers to a natural product that has a published chemical composition/structure with recognized biological activities/functions.

“Commercial database” refers to a service and/or a information management system that can be accessed by paying a subscribed fee. Such databases include, but are not limited to NERAC, DIOLOG, the Dictionary of Natural Products, Chemical Abstracts Service's Registration File, NAPROLERT, MEDLINE, DEREP, and the Bioactive Natural Product Database.

“Therapeutic” as used herein, includes treatment and/or prophylaxis. When used, therapeutic refers to humans, as well as other animals.

“Pharmaceutical or therapeutic profile” refers to the capability of modulating the activity and function of biological system, biochemical materials and gene targets without significant toxicity in the effective dose range.

“Pharmaceutically or therapeutically effective dose or amount” refers to a dosage level sufficient to induce a desired biological result. That result may be the delivery of a pharmaceutical agent, alleviation of the signs, symptoms or causes of a disease or any other desirous alteration of a biological system.

A “host” is a living subject, human or animal, into which the compositions described herein are administered.

“Safety profile” refers to the level to which an active nutraceutical and/or cosmetic agent allows maintenance of the normal activity and function of the biological system, biochemical materials, and molecular biology targets after it has been administered in a considerable amount.

A “standardized extract” refers to an extract generated from a production process that contains a specific component profile or fingerprint compounds with defined quantities of individual and/or total active natural products.

A “product candidate” refers to a standardized extract/fraction/natural product that possesses a desired biological activity and safety profile and is suitable as an commercial ingredient for the nutraceutical and/or cosmetic industries.

A “prototype product” refers to a trial product that is produced on manufacturing scale based on a specification of chemical profile and concentration of active agent from a designated biomass.

“Clinical evaluation” refers to studies of the effectiveness, safety, side effects, and contraindications on humans of a natural product based on a specifically designed and pre-approved clinical trial protocol.

Note, that throughout this application various citations are provided. Each citation is specifically incorporated herein in its entirety by reference.

The PhytoLogix™ discovery process can most generally be described as a comprehensive method for discovering and developing novel therapeutic pharmaceutical, nutraceutical and cosmetic agents comprising the steps of: (a) identifying and collecting a pre-identified biological sample; (b) extracting the biological sample using a two solvent system extraction procedure; (c) separating the extracts using two separate high throughput (HTP) fractionating methods and simultaneously determining the activity of each HTP fraction; (d) dereplicating the active fractions to identify the compounds present; and (e) generating an indication, pharmacological and safety profile for each novel compound from step (d). The pre-selection of sample to be collected is based upon traditional use.

To ensure that the PhytoLogiX™ discovery program was successful, it was essential to collect medicinal plants and other biosamples from around the world. Therefore, following the United Nations' Treaty of Convention on Biological Diversity, the Phytologix™ program has established eight international ethno-botanical collection agreements that cover the continents of Asia, South America, North America, Africa and other geological regions. In contrast to randomized plant collection programs, PhytologiX™ focuses on documented medicinal plants and other documented biomaterials. Due to thousands of years of historic use, these medicinal plants and other biomaterials have already been pre-selected and clinically tested for human consumption. Thus, they are most likely to yield safe and efficacious pharmaceutical, nutraceutical and cosmetic products in contrast to a randomized collection of biomaterials. Available information regarding historic use, in combination with available information provided by modem research on medicinal plants and other biomaterials provides considerable evidence regarding potential clinical indications, as well as, probable mechanisms of action. This information also assists in establishing screening models based on available ethnomedicinal information. The pre-selection based upon traditional use of plants and other natural biomaterials for Phytologix™ discovery is unique to this invention and critical to ensure a high positive hit rate, a safe product and a short discovery cycle. In a preferred embodiment, the sample is a medicinal plant.

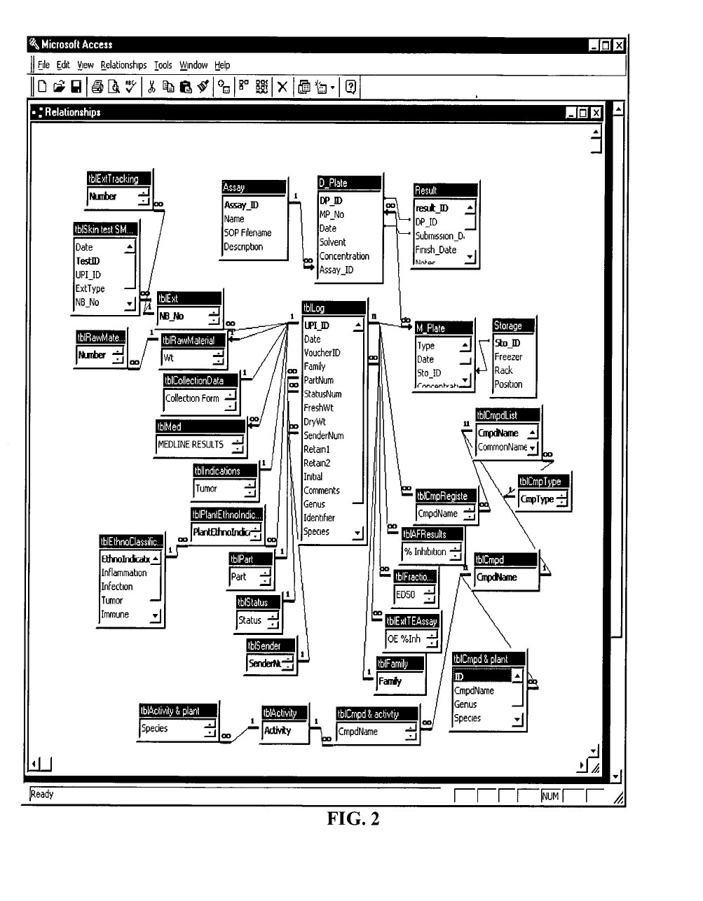

All sample collections were performed using the standardized collection and voucher specimen preparation procedures as illustrated in Example 1. In a preferred embodiment between 1 g and 10,000 g of sample are collected. A standardized plant/sample collection form was filled out for each sample collected and the information was transferred to a searchable informatics database as illustrated in Example 2 and FIG. 2. In one embodiment, this invention discloses a unique biomass registration system, which entails giving an exclusive code to each sample collected. The designated code is directly related to the natural origin of biomass as illustrated in Example 1, and can be used as a primary key to link all the information together in the informatic database.

Another embodiment of this invention includes the preparation of two specimen vouchers for each sample collected. A taxonomy form is attached to each voucher specimen for purposes of identification. The taxonomy form contains information regarding the identification of the sample, collection of the sample and collector name, etc. Such efforts are critical and unique to guarantee the integrity and authenticity of the biomass during the research stage of the process and ensure the potential of successful recollection and production during the production stage of the invention. As set forth in Example 1, to date the Phytologix™ collection process has resulted in the acquisition of 1,170 medicinal plants and other natural materials. Two sets of voucher specimens have been prepared for each sample acquired as described in Example 1. Additionally, 500 to 2,000 grams of dry materials per biomass have been stored. This collection of specimens includes 266 families, 805 genera and 932 different species collected from around the world.

The present invention includes a Biolnformatics driven assessment of a novel medicinal plant library. With a current collection of more than one thousand and potential access to more than 10,000 medicinal plants and other biological specimens throughout the world, the Phytologix™ discovery program includes a relational database containing information including, but not limited to ethno-indication and phytochemistry. This database enables the prioritizing for screening of medicinal plants having the most potential based upon traditional use. An example of this is demonstrated in Table 1, using for purposes of illustration the goal of the discovery and development of a novel nutraceutical product for arthritis pain. To do this, one would perform a search of the informatic database using “Arthritis” as a key word. The search results in the listing of 18 plants that have been used traditionally for the treatment of arthritic pain. (Table 1) The discovery process can therefore be focused on the evaluation of those eighteen plants, as opposed to a randomized screening of any plants. This strategy offers a significant advantage over randomized screening in that screening methods traditionally use animal models and thus, only a limited number of samples can be screened. However, randomized screening is not excluded according to the method of this invention. The method of this invention can also be extended to high throughput screening methodology, based on mechanism of action, as well as, traditional use. This invention also includes the alternative of random screening by offering standardized extracts and HTP fractions in 96-deep-well plates.

The PhytoLogix™ Discovery Process relies upon multiple standardized extraction and fractionation protocols, which allow the generation of diversified extracts and fractionation libraries in a high throughput format at a limited cost. Every biomass collected in the Phytologix™ program was processed following a standardized extraction protocol, as describeda in Example 3. This method of extraction offers several advantages when compared to the extraction methodology described to date. First and foremost, the dual extraction strategy described herein, provides a significantly more complete and extensive natural product profile from each biomass. Not a single important type of natural product will be missed using this process.

As described in Example 3, the sample, preferably from 1 g to 1000 g, is first extracted with a medium polarity solvent combination, such as methylene chloride:methanol in a ratio of 1:1. The combination of a low polarity solvent, such as methylene chloride with a solvent of high polarity, such as methanol will yield a solvent system that can dissolve not only low to medium polarity compounds, such as terpenoids, alkaloids, fatty acids, flavonoids, steroids, lignans, benzophenones, chromones, and anthraquinones, but also can dissolve high polarity compounds, such as terpenoids, alkaloids, fatty acids, flavonoids, steroids, lignans, benzophenones, chromones, and anthraquinones etc., which contain multiple polar functional groups and/or mono-, di- and tri-glycosides. The low polarity solvents can be selected from any known low polarity solvents used in the art to perform extractions. In a preferred embodiment the low polarity solvent is selected from the group consisting of an alkane having 6-10 carbons, a halogenated alkane having 1-4 carbon atoms, wherein each carbon atom has 1-4 halogen atoms, an ester having the formula, R′COOR″, wherein R′ is selected from an alkyl group having between 1-6 carbons and R″ is selected from an alkyl group having between 1-8 carbons and a ketone having between 3-12 carbons. Examples of low polarity solvents include, but are not limited to, methylene chloride, ethyl acetate and chloroform. The polar solvent can also be selected from any known polar solvents used in the art to perform extractions. In a preferred embodiment the polar solvent is selected from the group including, but not limited to, DMSO, THF and an alcohol having one to eight carbons. Examples of alcohols include, but are not limited to methanol, ethanol, propanols and butanols. Water soluble, higher polarity components, such as quaternary and ionized alkaloids, oligosaccharides, polysaccharides, salts of organic acids, phenolic salts, anthrocyanidins, amino acids, peptides, tannins, minerals and other inorganic compounds, will only be extracted by water, acidic water, basic water, or aqueous buffer. Therefore, following extraction with the dual organic solvent system, the biomass is extracted with water, acidic water, basic water, or aqueous buffer to dissolve the water-soluble components contained in the biomass. In a preferred embodiment, the quantity of solvents used in both extractions is one to ten times the ratio of the weight of the extracted sample. The extraction may be carried out using any known methods for extraction including, but not limited to shaking, sonication, refluxing, stirring, and pressurized mixing, and filtering. Representative organic and aqueous extracts performed on various plant species are set forth in Table 2.

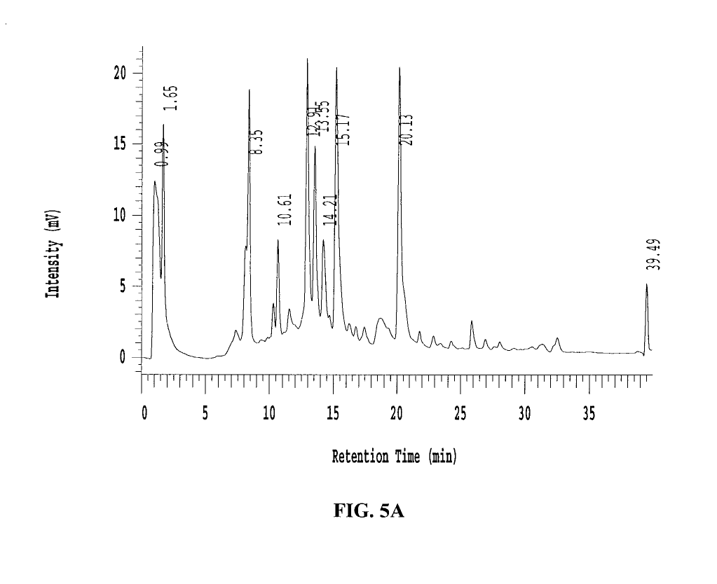

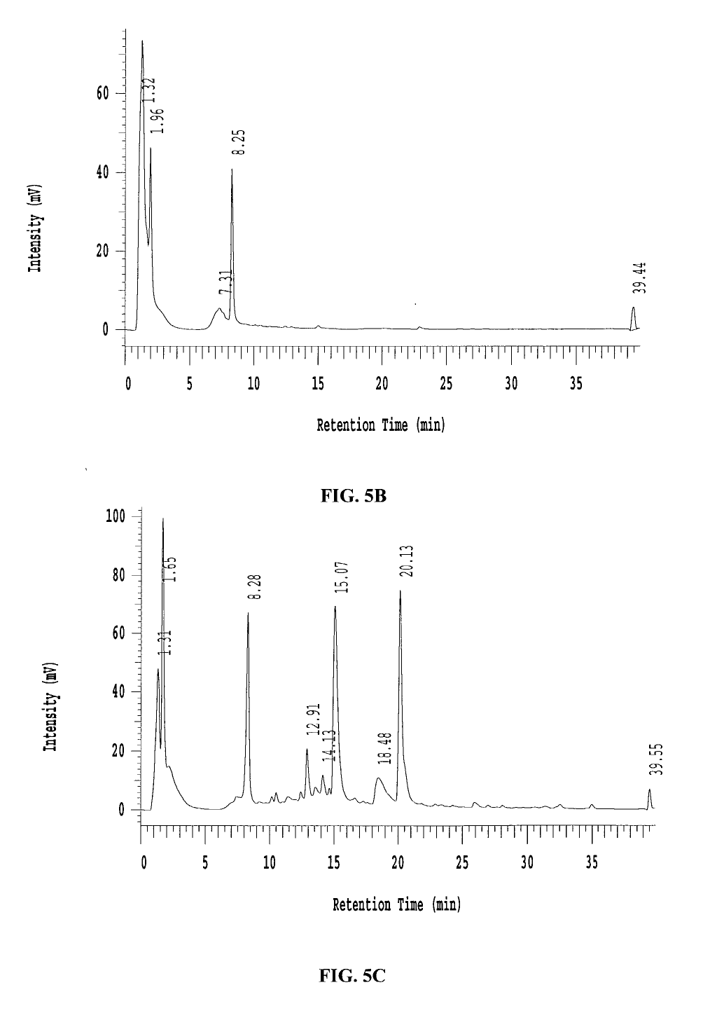

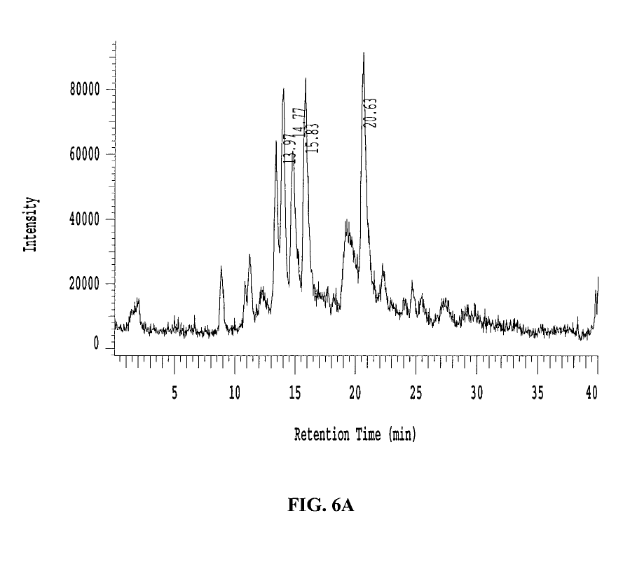

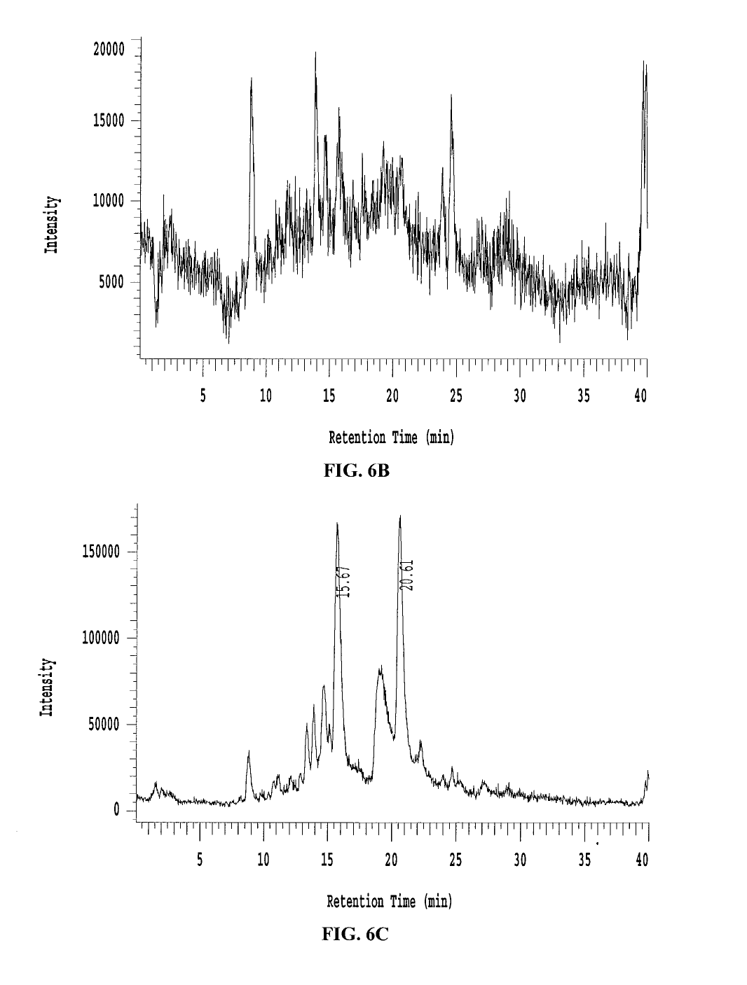

The efficiency of the extraction methodology described herein is illustrated in Example 4. Further extraction of the biomass with methanol after the organic and aqueous extractions described in Example 3, provided only a small amount of extractible material (Table 3), having exactly the same HPLC chromatograms (FIGS. 5 and 6). The HPLC chromatograms depicted in FIGS. 5 and 6 were generated using two different detection methods. Photo Diode Array (FIG. 5) and ion trap mass spectrometer (FIG. 6). As can be seen in FIGS. 5 and 6, using either method of detection there were no specific peaks present in the methanol extract, that were not also present in either the organic extract or the aqueous extract.

Another advantage of the extraction methodology described herein is that the extraction process yields enough material for further fractionation and bioassays. For example, extraction of 60 grams of biomass, generates approximately 1-8 grams of organic extract and 1-6 grams of aqueous extract. These quantities provide enough material for a number of screens and HTP fractionations.

In one embodiment of this invention, a novel method to prepare an extract library for high throughput assays is described. This method comprises the generation of a set of extract master plates, by dissolving the organic and aqueous extracts in DMSO and deionized (DI) water, respectively, at a concentration of between 0.01 mg to 1000 mg/mL of solvent. In a preferred embodiment the concentration of the extract is 50 mg/mL of solvent. The sample master plate is selected from the group including, but not limited to, a 96, 192, 384, 576, 768, 960, 1152, 1344 or 1536 well plate. In a preferred embodiment the solutions were stored in a 96-deep-well plate with 88 samples per plate. The extracts can then be aliquoted and screened with high throughput models. There is enough material in each cell to complete 50-100 typical high throughput screens. Other solvents that can be used to dissolve the organic and aqueous extracts include, but are not limited to DMSO, DMF, THF, ketones having three to ten carbons and alcohols having one to five carbons.

A significant discovery disclosed herein is a novel method for the chromatography or high throughput fractionation of the extracts, which is both efficient and economically sound. This method is described in Examples 5 and 6. The method for the high throughput fraction of extract is comprised of the steps of: (a) using a parallel chromatography system or a high throughput purification (HTP) system; (b) separating the organic extract with a normal phase pre-packed column; (c) separating the aqueous extract with a reverse phase pre-packed column; (d) detecting the eluent with detector(s); (e) collecting fractions; and (f) evaporating the solvent. In a preferred embodiment the chromatography system is comprised of two to four solvent delivery pumps, solvent mixers, and appropriate auto line switchers. The chromatography is carried out at ambient, low, medium or high solvent pressure and at ambient temperature or a temperature from 20 to 80° C. The normal phase column is packed with a resin selected from the group including, but not limited to silica gel, alumina, and amino propyl, cyano propyl, diol florisil or polyamide, ion exchange group-bond resins. The reverse phase column is packed with a resin selected from the group including, but not limited to a C-2, C-4, C-8, C-18, LH-20, XAD-4, XAD-16 or polystyrene-divinyl benzene based resin. The particle size of the resins is from 10 to 200 μm. The chromatography column is packed with 1 to 500 grams of resin.

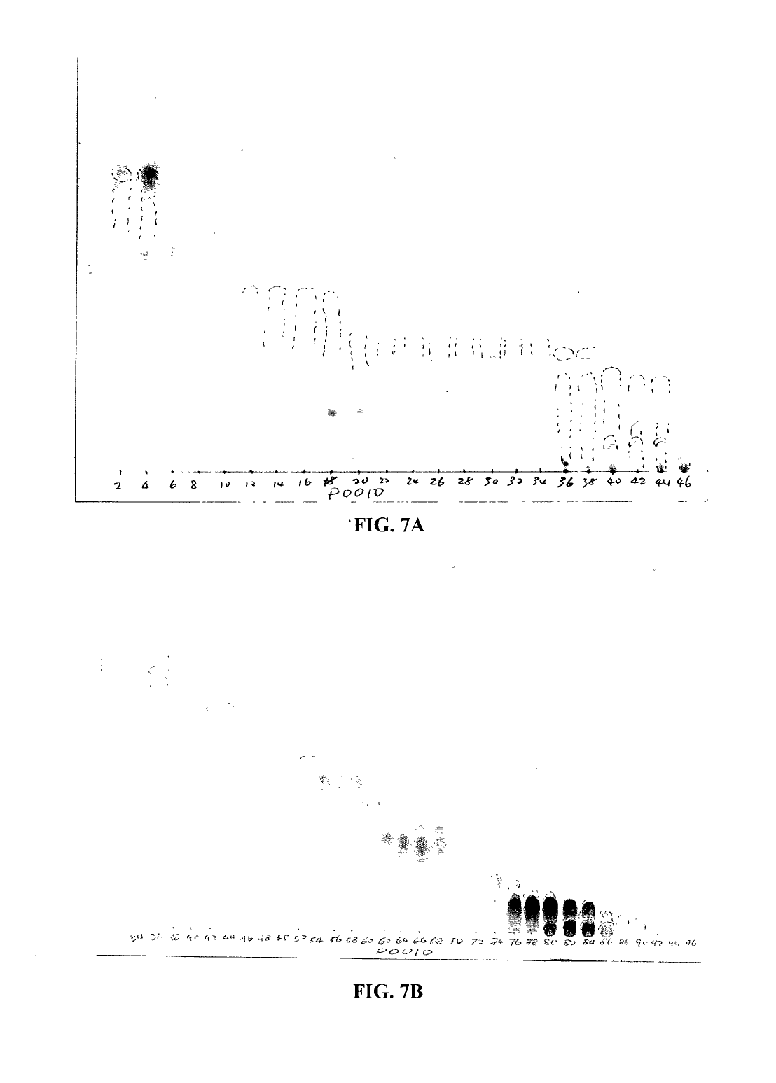

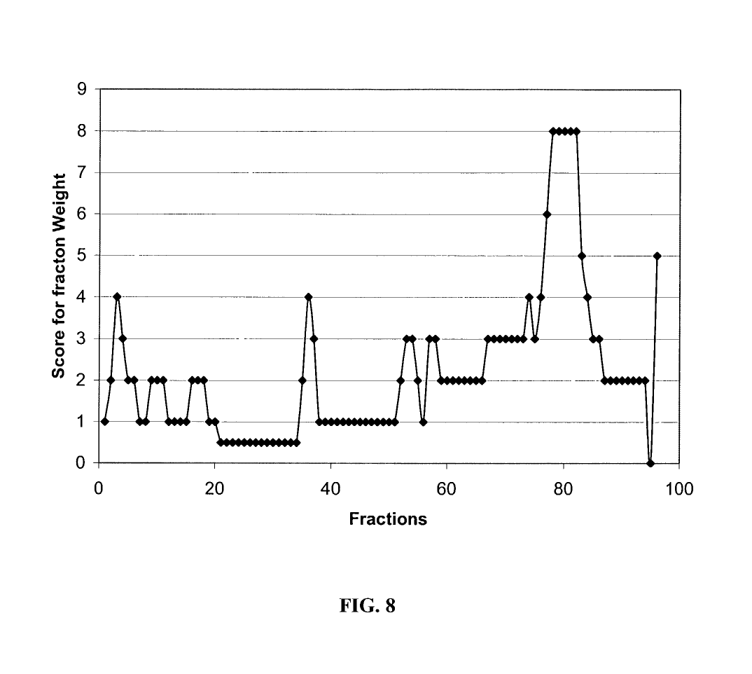

Many different methods have been reported for the fractionation of plant extracts. Some of those methods even utilize solid phases similar to those described herein, such as silica gel and reverse phase C-18 columns. However, as set forth in the Background of the Invention, most of the prior art methods use step gradients to provide a limited number of fractions (usually less than 20 fractions) and incomplete separations that require further chromatographic purification. The present invention is superior to prior art methods, in that the separation on the normal phase column is carried out using a gradient of a unique combination of three organic solvents that include an alkane having from six to ten carbons, an ester R¹COOR², wherein R¹ is selected from an alkyl group having between one to five carbon and R² is selected from an alkyl group having between one to six carbons, and an alcohol (R³OH) wherein R³ is an alkyl group having between one to six carbons. This three-solvent system combination significantly improves separation and the quality of fraction in each well, as illustrated in FIGS. 7 and 8. As demonstrated by this invention, from the organic (Example 5) and/or aqueous extracts (Example 6), a natural product can be purified using a single column. Furthermore the product is distributed in limited number of cells/fractions (usually in 2-8 cells). The separation on the reverse phase column is carried out with a combination of two solvents: DI water and a solvent selected from the group consisting of an alcohol with one to four carbons, acetonitrile, THF, or a ketone having three to twelve carbons.

Although, some known methods use HPLC systems with gradient capacity and better separation capability, the quantity of the materials that can be loaded on the columns and the throughput of the fractionation are incomparable with the current invention. As illustrated in Examples 5 and 6, the organic and aqueous extracts can be loaded onto commercially available pre-packed columns, typically, a silica gel column for organic extraction and a C-18 column for aqueous extraction, at a level of 100 mg to 2000 mg. At such levels, each fraction resulting from the high throughput purification will contain milligrams of materials that can be dissolved into a solution at concentrations of 1-10 mg/mL. Thus, this invention has solved two of the major problems in natural product research, one of which is how to prevent false negative results, in which the minor active, but rather novel compounds fall under the bioassay detection limits or positive threshold. The other problem solved is how to eliminate false positives due to synergistic effects from a mixture of multiple compounds with lower than desirable biological potency. The method disclosed herein not only separates individual components present in the crude extracts, but also significantly enriches minor active components in the plant extracts, which leads to a much greater chance that these minor components will be detected in the screening process.

The detector may be any detector used in the art for such purposes including, but not limited to an ultraviolet (UV)/visual light detector, a Mass Spectrometer (MS) detector, a Nuclear Magnetic Resonance (NMR) detector, a reflex index (RI) detector or a light scattering detector (LSD). The ultraviolet (UV)/visual light detector may be comprised of single or dual channels with single, continuing or broadband wavelength from 100-1000 nm. The MS detector may be comprised of an electronic spray ionization, sonic spray ionization or chemical ionization chamber; ion trap or single or triple quadruple mass detection with positive or negative mode. The NMR detector may be comprised of a proton or carbon probe.

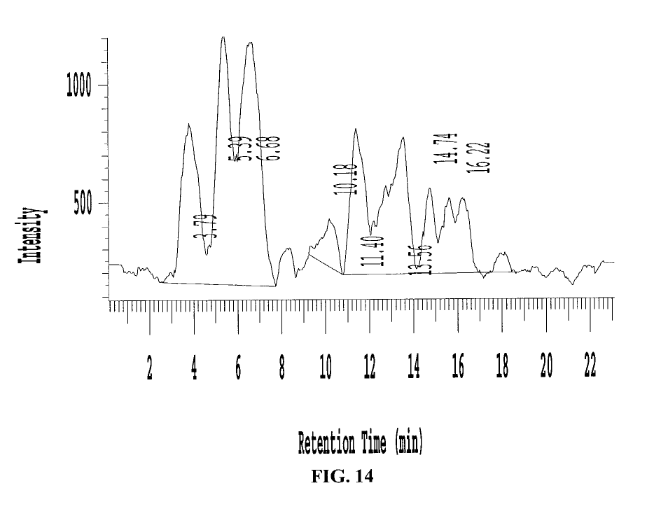

Another unique characteristic of this invention is the online structure information collection. This invention utilizes a high-pressure chromatography system in a parallel processing mode, i.e., multiple simultaneous column runs coupled to a robot controlled liquid handling system that is triggered to deliver chromatographic eluent (containing individual chemical compounds) based upon a pre-programmed time or volume quantity, or on the basis of a chemical response pattern, preferably an ultraviolet light absorption spectrum or ionization pattern. This pattern, when compared to a library of patterns by computer analysis will determine whether the compound is a known or unknown chemical entity. FIGS. 14 to 16 depict the online mass spectroscopic data of one HTP fraction and the offline analysis of the same fraction. This method is much more efficient, because the spectroscopic data is collected at the time of separation, rather than analyzing collected fractions in a separate process. Additionally, the method has been shown to be just as accurate. In the example illustrated in FIGS. 14-16, the HTP system directed the sample simultaneously to both the liquid handling system where an aliquot of the eluent was dispensed in microtiter plates and to an ion trap mass spectrometer with a super sonic ionization chamber where the molecular ion and fragmentation pattern of the compound were determined. From the mass spectrum, it is possible to derive the molecular weight and general structural information regarding the components of the fractions. This information is compared to a chemical library by computer analysis to confirm purity and tentative identification.

As demonstrated in Examples 5 and 6, the method disclosed herein is proven to be highly efficient. The throughput of the fractionation process can generate 1232 fractions daily from 14 organic extracts or 2618 fractions from 32 aqueous extracts. This throughput is ten times higher than any of the known methods described as set forth in the Background of the Invention.

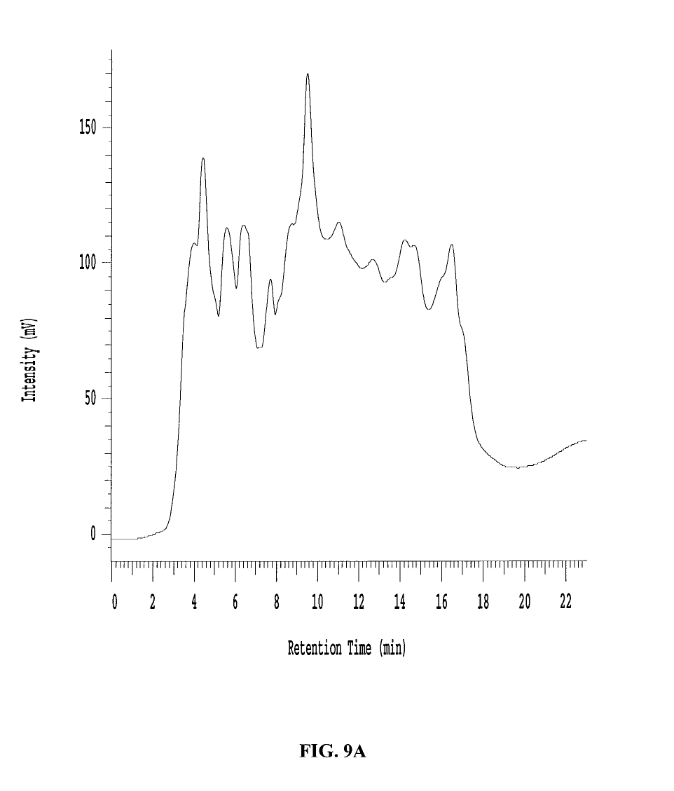

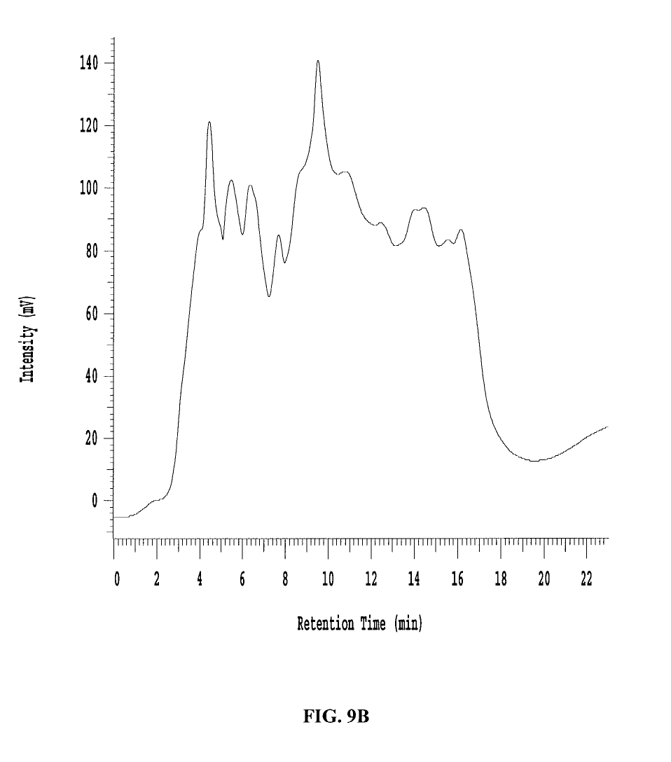

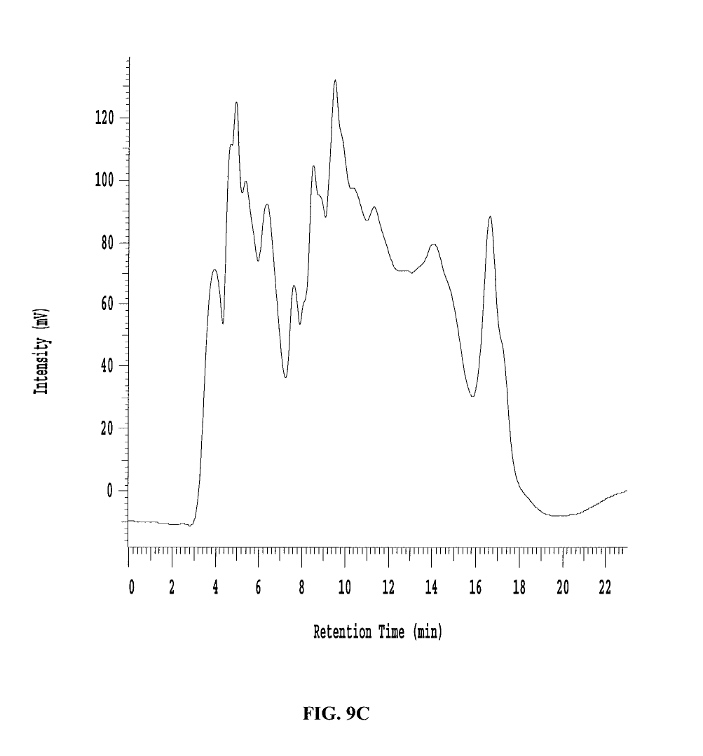

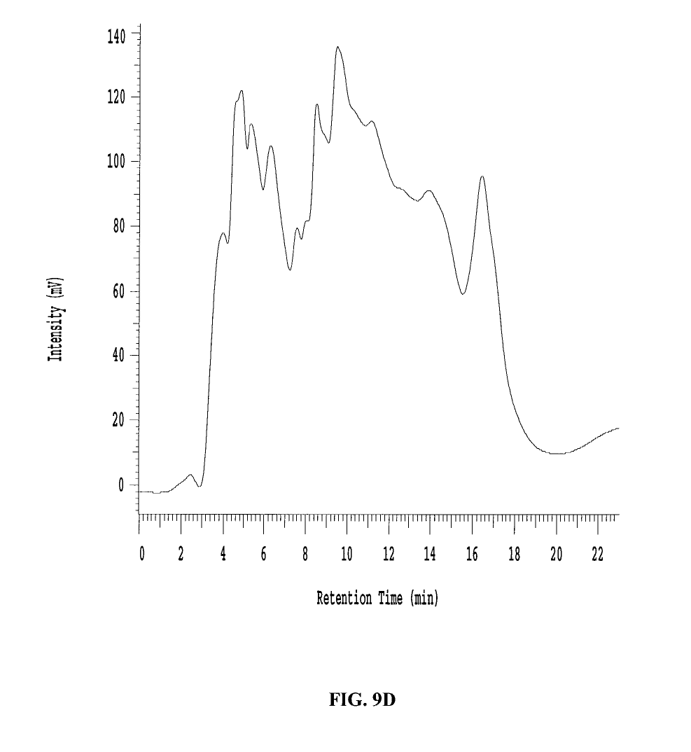

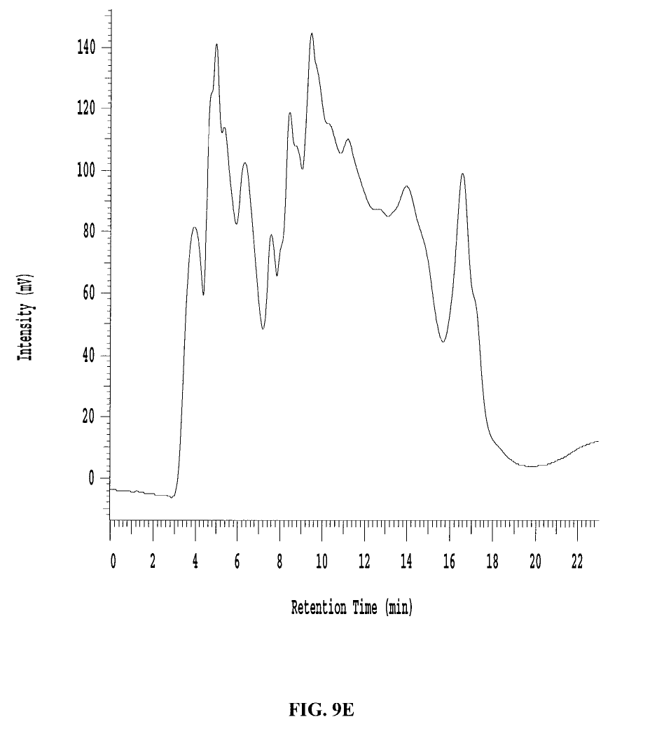

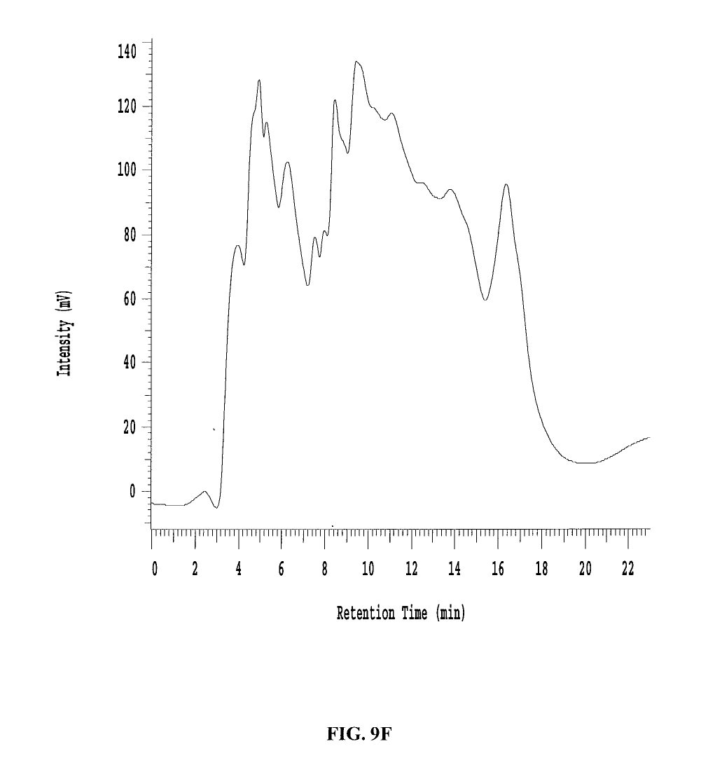

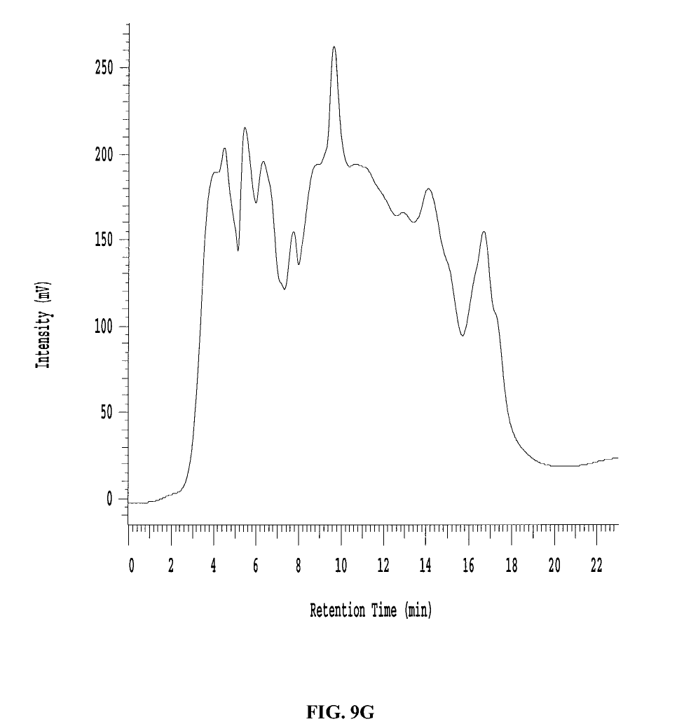

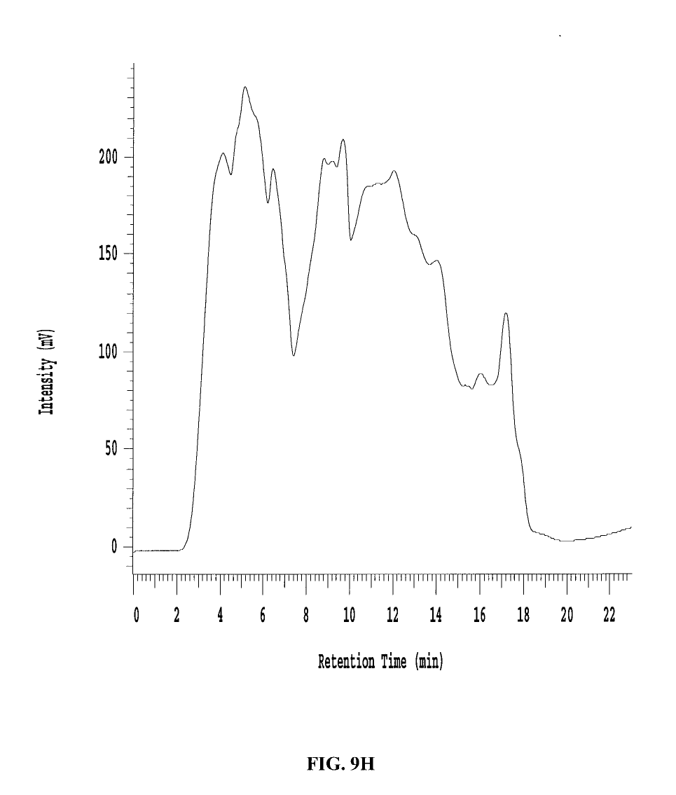

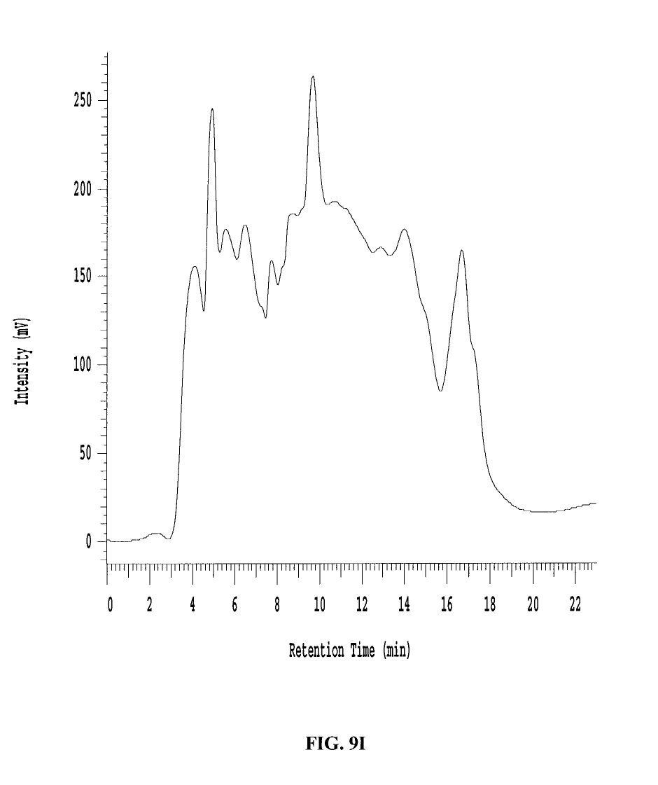

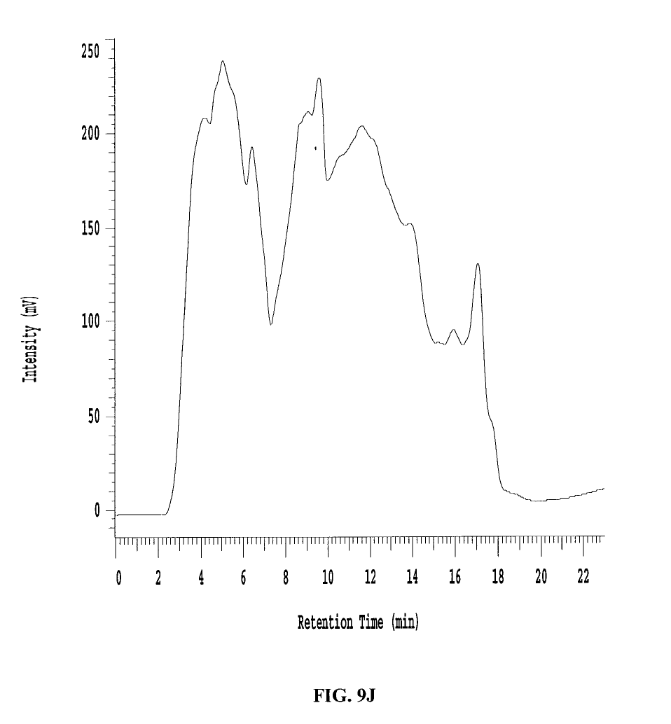

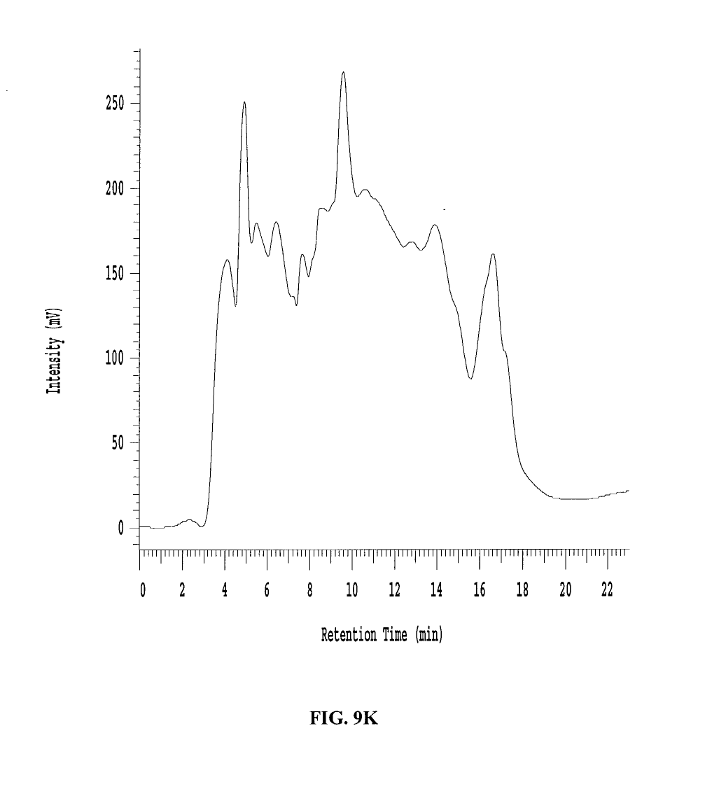

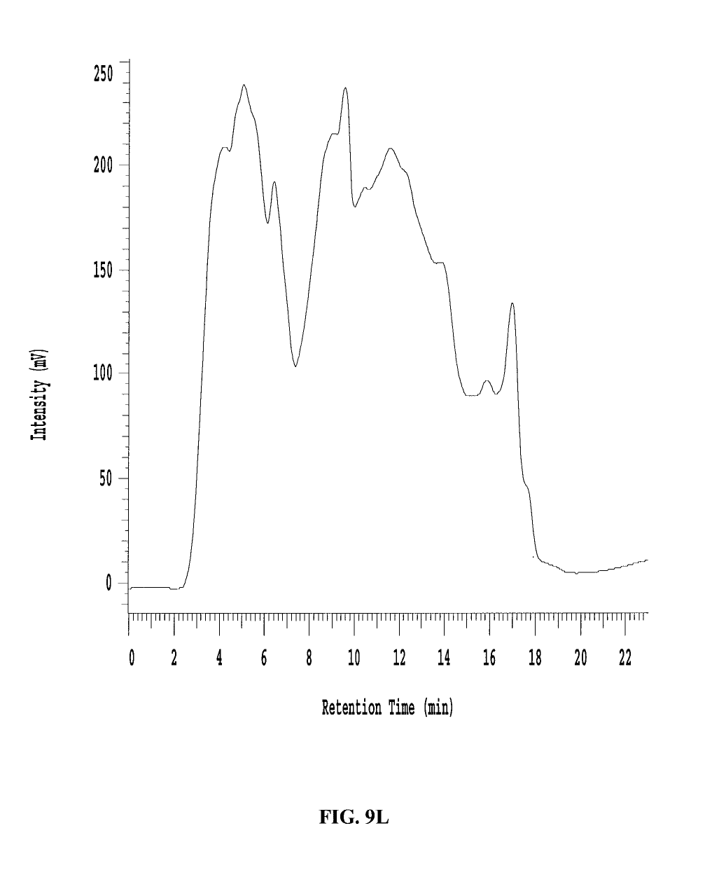

Finally, a significant advantage of the methodology disclosed herein is the low cost of operation. The detailed analyses of the cost of consumables are set forth in Tables 4 and 5. The material costs to generate one fraction of a sample from the organic or aqueous extracts are only sixteen cents and thirty-two cents, respectively. The normal phase columns can only be used one time, however, the reverse phase columns can be reused up to sixty times with appropriate washing between each run. The performance of the C-18 column has been closely monitored with a known compound mixture of aloe chromones (data not shown). The separation was shown to be highly reproducible as demonstrated in FIG. 9, by performing twelve C-18 column fractionations on same aqueous extract. There is no comparable method in the prior art, which can generate such a high quality natural product fraction library at such a low cost and high throughput.

Once the biological and/or indication targets are defined, the PhytoLogix™ approach to implementing a high throughput screen (HTS) is accomplished by applying biochemical (enzymatic, receptor binding assays), gene expression, functional or biological models as the primary means of screening extracts to identify compounds in the extract having a particular activity. In a preferred embodiment the model used includes, but is not limited to, enzyme inhibition, receptor binding, gene expression, cell function regulation, protein production, animal physiological, neurological, and behavior function regulation and animal disease model manipulation and other measurements of biological function which are known to those in the art. The data regarding the activity of the fractions can be collected from the extracts, fractions of the extracts, purified compounds, semi-synthetic and synthetic compounds.

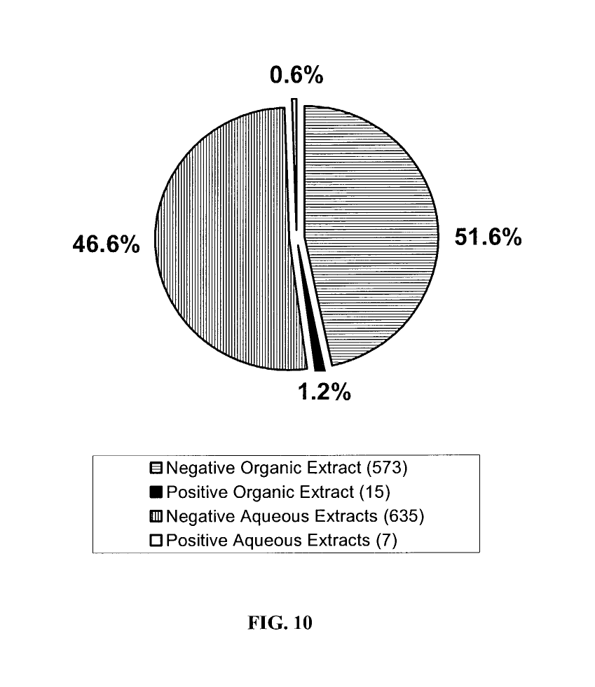

To demonstrate the value of an extract library generated using the method of the present invention, Example 7 describes an enzymatic screening and the results obtained. In order to identify anti-inflammatory compounds whose mechanism of action is the inhibition of the cyclooxygenase (COX) enzymes, a COX enzyme inhibition assay was developed to evaluate an extract library comprised of 1230 extracts from 615 medicinal plants collected from China, India, and other countries. The general method used for preparing these extracts is described in Example 3. The extraction process yielded an organic and an aqueous extract for each species examined. These primary extracts were the source material used in the preliminary assay to identify inhibitors of the enzyme's peroxidase activity, which is one of the main functional activities of cyclooxygenase and is responsible for inflammation by the conversion of PGG2 to PGH2 and ultimately PGE2. This assay is described in Example 7 and the results are summarized in FIG. 10. With reference to FIG. 10, after screening 1230 plant extracts, a total of 15 organic extracts (1.2%) and 7 aqueous extracts (0.6%) were confirmed as having greater than 60% inhibition with a dose response confirmed by separate experimentation as described below. The representative activity measurements on individual plant extracts are set forth in Table 6. With reference to Table 6, it can be seen that two species of Scutellaria and three other plant species, all of which contain Free-B-ring flavonoids as common components, showed inhibitory activity in the primary screen against the peroxidase activity of COX-2 albeit to differing degrees. The COX-2 inhibitory activity is found predominantly in the organic extracts, which contain most of the medium polarity Free-B-Ring flavonoids. The COX-2 inhibitory activity from the primary assay of the crude extracts was confirmed by measurement of dose response and IC₅₀ (the concentration required to inhibit 50% of the enzyme's activity). The IC₅₀ values are set forth in Table 7. As can be seen in Table 7, in this assay Scutellaria orthocalyx root extract and Murica nana leaf extract were the most efficacious (IC₅₀=6-10 μg/mL). Extracts from Scutellaria sp. that demonstrated the greatest selectivity against COX-2 relative to COX-1 were those generated from Scutellaria lateriflora (COX-2 IC₅₀: 30 μg/mL; COX-1 IC₅₀: 80 μg/mL). Thus, the primary screen for inhibitors of the COX enzyme resulted in the identification of twenty-two extracts that were efficacious in vitro and some of which demonstrated specificity for the COX-2 enzyme relative to COX-1.

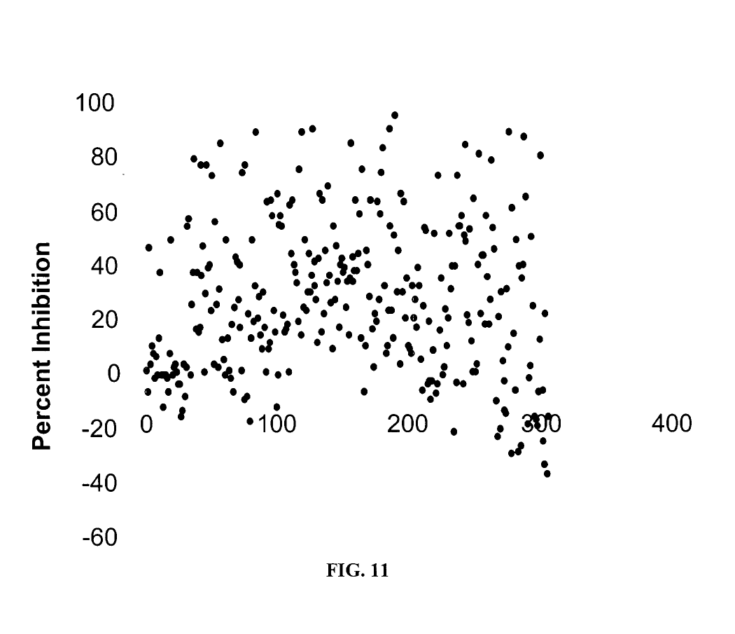

Example 8 illustrates the screening of a plant extract library for inhibitors of the enzyme tyrosinase in an attempt to identify a novel skin whitener for cosmetic use. From this assay, 43 organic extracts were identified as having tyrosinase inhibitory activity, equivalent to a hit rate of 5.6% hit rate. This was significantly higher than the 0.78% hit rate for the aqueous extracts, based on the screening of 774 plant extracts. The results are set forth in FIG. 11. Since the targeted indication is a cosmetic product for use as a skin whitener, the compounds with lower polarity should have better skin penetration. The screening results demonstrated the quality of the extract library that automatically eliminated the natural products with unwanted physical properties due to the selectivity of both the extraction and bioassay processes.

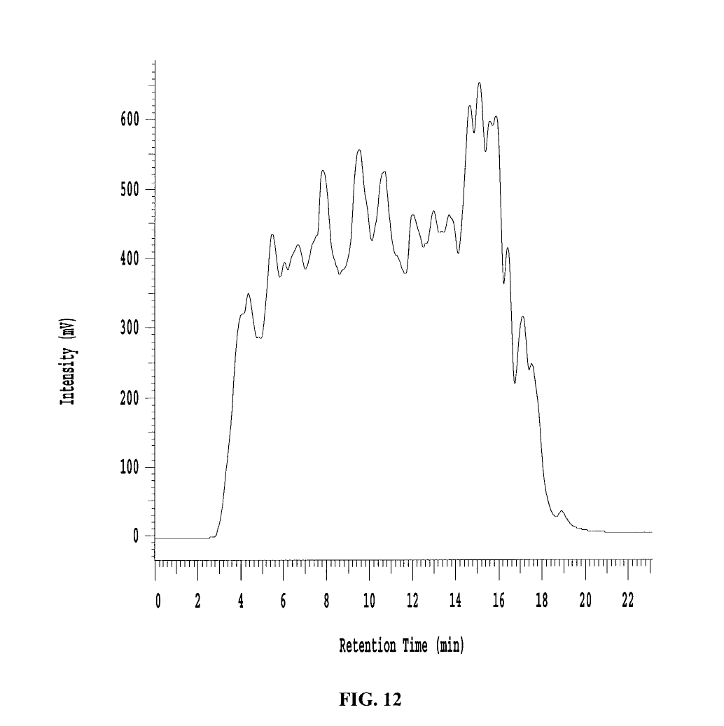

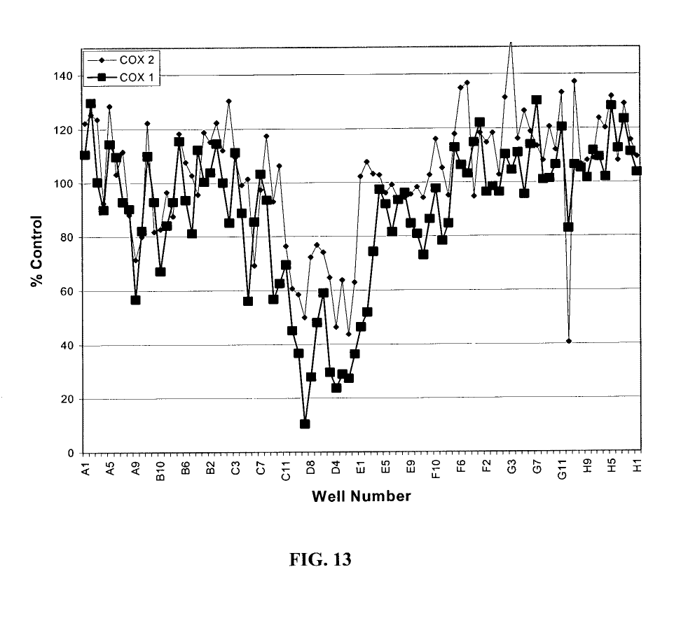

Direct screening of the fractionation library has its own value, since each fraction will contain one major compound in high enough concentration that the likelihood of obtaining false positives and false negatives, which is commonly a problem with crude extracts, will be eliminated. Additionally, minor bioactive components are more likely to be detected, because the concentration of these components is enriched enough to render them detectable. Example 9 describes the screening of the bioactive extracts isolated as described in Example 7. In this example each of the HTP fractions was examined for its ability to inhibit the peroxidase activity of both COX-1 and COX-2. A representative HTP/UV chromatogram of the fractions derived from the aqueous extract of Camellia sinensi is illustrated in FIG. 12. After screening all of the 88 fractions from the 96-well plate, a total of 8 HTP fractions exhibited greater than 60% COX inhibition as illustrated in FIG. 13. Following the dereplication process described in Example 11, ten individual compounds were identified in those fractions and surrounding fractions that contributed to the COX activity. There are many components in the crude aqueous extract that could interfere with the assay or cover up the potency. However, in this invention, these components have been separated out into other wells. This process greatly enhances the positive hit rate from a single data point in the crude extract to eight positive fractions from which multiple bioactive compounds have been identified.

One of the major advantages of the HTP fraction library created using the method disclosed herein is the significantly improved efficiency and accuracy of the dereplication. Dereplication is a method used to identify to the greatest extent possible, the structure and physical property profile of an active sample in order to determine the likelihood of the existence of novel compounds in the sample. The determination that there may be novel compounds justifies further isolation and identification efforts. To achieve this goal, an internal structure and spectroscopic characteristics database was developed with more than 250 known pure compounds that possess representative structural skeletons of common natural products. Example 10 describes the method used to construct this database. As illustrated in Example 10, the HPLC method used for the analysis of these compounds was an improvement over known methods. The method is much shorter (total of 8.5 minutes per analysis) without sacrificing separation capacity, as a result of using a smaller particle size C-18 resin, a smaller diameter, but a longer column. This internal database currently contains six fields for each individual compound including, type of compound, name of compound, molecular weight, chemical structure, UV spectrum and retention time. Table 8 sets forth representative information in the database for flavonoids, alkaloids, caffeic acids, terpenoids, chromones, anthraquinones, iridoids, acetophenones, and coumarins. Using the standardized HPLC method described above, an active sample will be separated with a reverse and/or normal phase column with a gradient solvent system. The detected peak from PDA and MS will be analyzed as follows: the UV spectrum of the peak is searched against the internal spectrum database and external database for structural skeleton or the type of compound, i.e., flavan, isoflavonoid, terpenoid, caffeic acid derivative etc.; the molecular ion of the peak is then used for initiating a molecular weight search using a database, such as the Dictionary of Natural Products, with other searchable fields, such as, plant Latin name, type of compound, UV spectrum; and finally the retention time is used to get a general idea about the polarity, log P, solubility, and other physical properties of the compound.

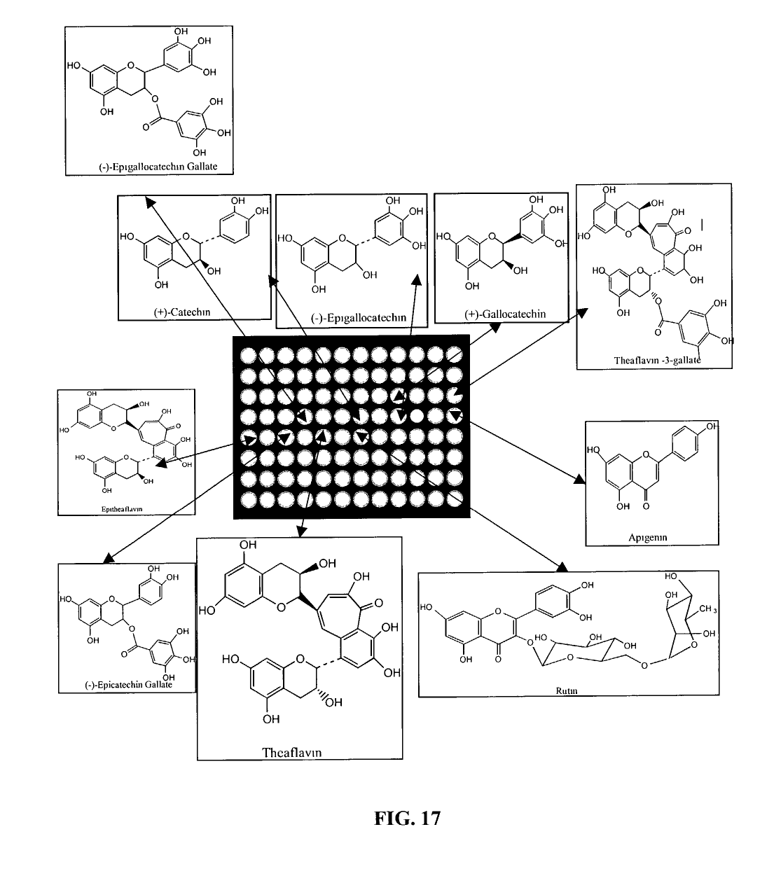

The uniqueness of the Phytologx™ dereplication process is illustrated in Examples 10 and 11. Example 11 describes the dereplication of the HTP fraction library derived from the aqueous extract of green tea for inhibitors of COX peroxidase. A total of 24 fractions surrounding the COX inhibition peaks as shown in FIG. 13 were analyzed using standardized HPLC. After obtaining and evaluating retention times, UV and MS data, all of the major components in each of the 24 cells have been dereplicated and identified as known catechin and flavonoid types of compounds. The results are set forth in FIG. 17. Each compound was distributed among 3-4 individual cells. Since the COX inhibitory activity of catechins and flavonoids are well known, the conclusion from the dereplication process is that these active fractions are not worth pursuing.

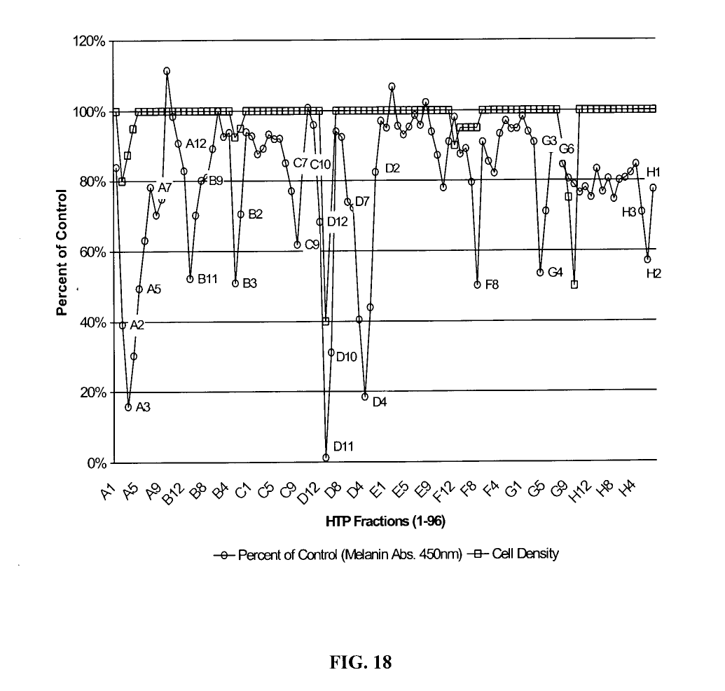

Example 12 describes the results of the dereplication of an HTP fraction library for inhibition of melanin formation in a B16 cell line. Briefly, following the inhibition and cell viability assay, the active organic extract from the whole plant of Mallotus repandus was fractionated with HTP as described in Example 5. All of the HTP fractions were tested for tyrosinase inhibitory activity and the results are set forth in FIG. 18. With reference to FIG. 18, there are three major peaks exhibiting >50% inhibition of melanin synthesis and seven other peaks exhibiting weaker inhibition. The sharp activity peaks are indicative of the quality of the separations, which distributed the active components in three to five cells.

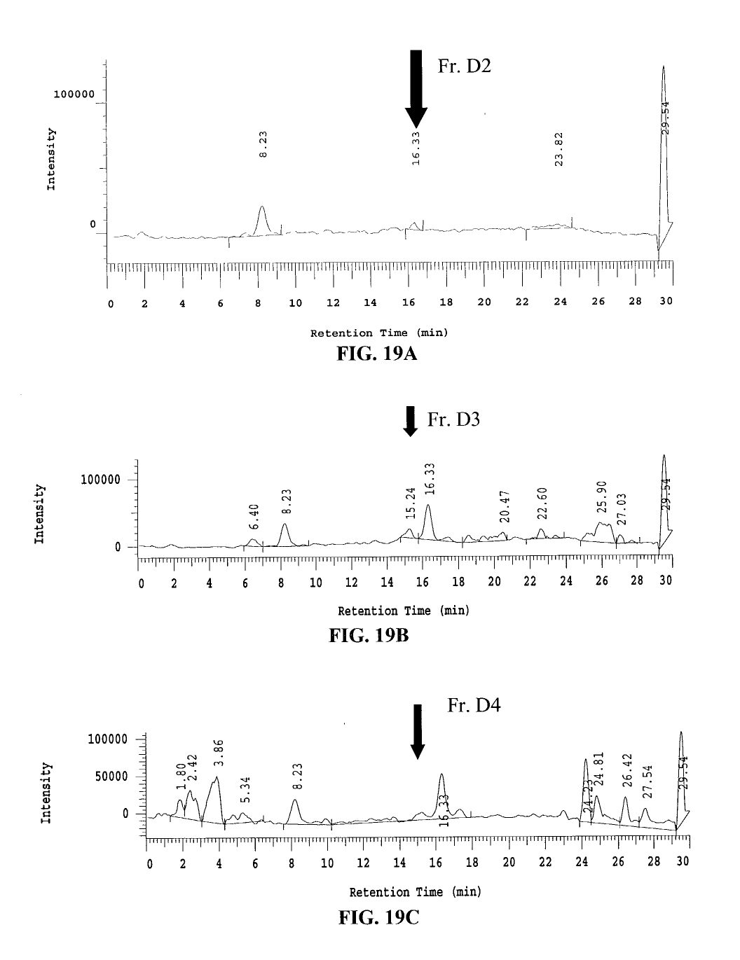

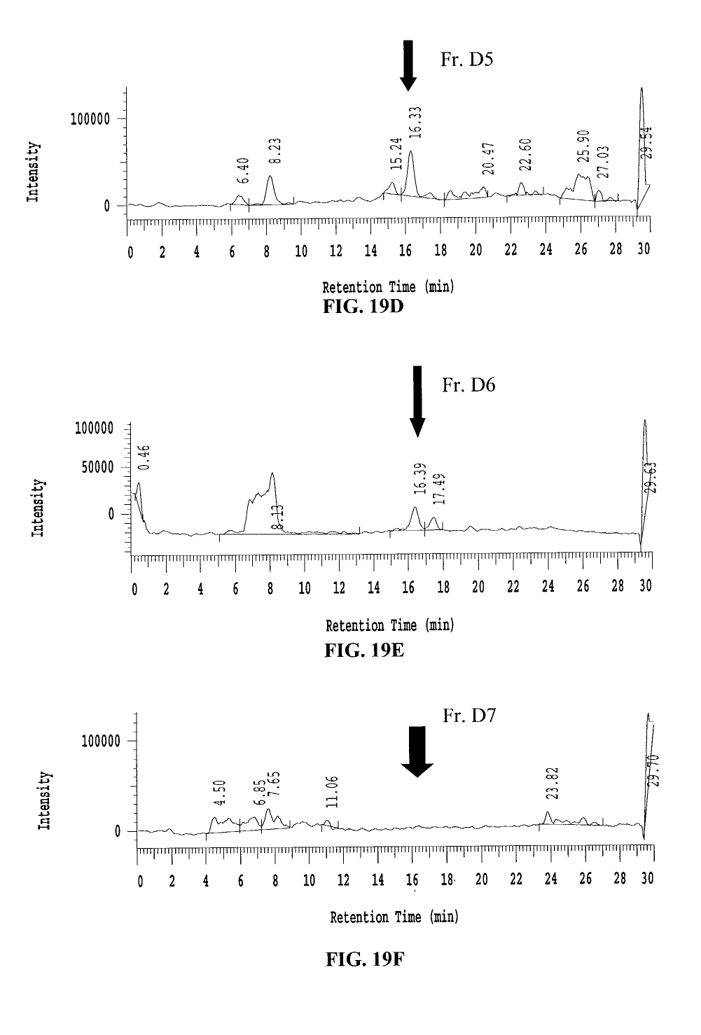

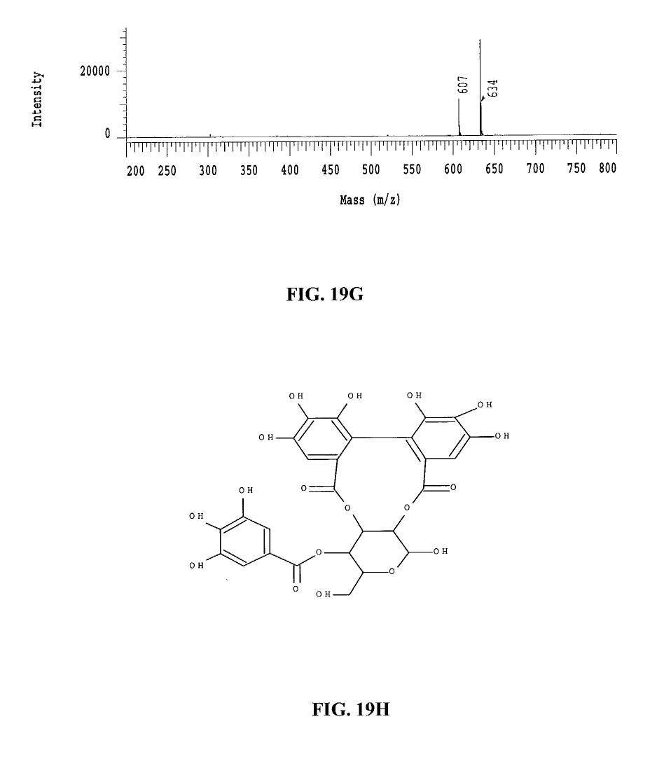

Since the melanin formation assay was run against a cell viability assay, the activity peak maximum at fraction D11 is most likely due to cytotoxicity. The dereplication of another active peak located from fractions D2 to D7 is illustrated in FIG. 19. Every active fraction was analyzed by HPLC. There was a peak located at Rt=16.33 minutes in the HPLC chromatogram of each fraction. This peak showed the same increasing to decreasing intensity as the trend exhibited by the melanin inhibition activities of those fractions. Further analysis of the UV spectrum revealed that this compound was a gallic acid derivative. Search of the Dictionary of Natural Products for molecular ion and plant genus name lead to the identification of a known compound—Pterocaryanin B, whose structure is depicted in FIG. 19H. The poly-hydroxyl groups in the structure are what are responsible for the inhibition of melanin synthesis. Because this was a known compound no further isolation was necessary.

In conclusion, the accelerated active identification process, referred to as dereplication, which includes an internal Structure and Spectroscopic database, in conjunction with use of the Dictionary of Natural Products and other external databases accessible through NERAC service, provides highly efficient and rapid structural identification that enables elimination of known components, false positives and false negatives and leads to the discovery of the novel active natural products by performance of assay directed isolations. The methodology described herein offers significant advantages over known methods, particularly the development and use of the purified compound library. First, with the unique separation conditions and a single chromatography approach, the Phytologix™ HTP fraction library is much easier and cheaper to generate than other known libraries as described in the Background of the Invention. As demonstrated, the Phytologix™ HTP library contains high purity natural products in individual cells in a sufficient quantity to execute a number of high throughput assays. Second, the dereplication process according to the Phytologix™ platform is closely related to the bioassay results. Thus, only the active fractions and limited surrounding fractions are analyzed, which both saves time and focuses the effort, as opposed to dereplication of all fractions and/or randomized dereplication of some fractions as described in the prior art. Third, Phytologix™ dereplication utilizes the natural weight distribution curve of the active fractions, obtained from the UV or MS chromatograms by matching with biological activity profiles, enables identification of the active components much more accurately and quickly. Finally, it has been demonstrated that the shorter offline HPLC method and online data collection from the Phytologix™ process can achieve the same results and conclusions in a much more cost effective and time efficient manner.

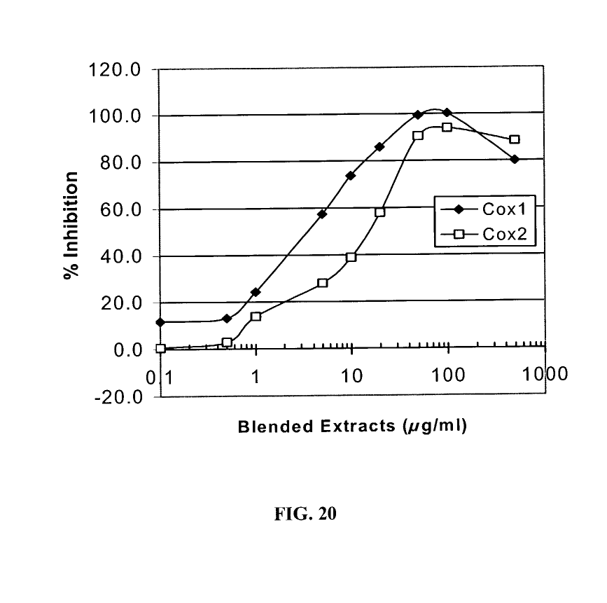

If it is determined from the dereplication process that the active HTP fractions contain a novel compound or compounds, an extensive isolation, purification and identification process will be initialized, as illustrated in Example 13. This example illustrates the isolation, purification and identification of the compound Baicalein, which inhibits the activity of the COX enzyme. Once purified the anti-inflammatory activity of the pure compound was confirmed. The results are set forth in FIG. 20.

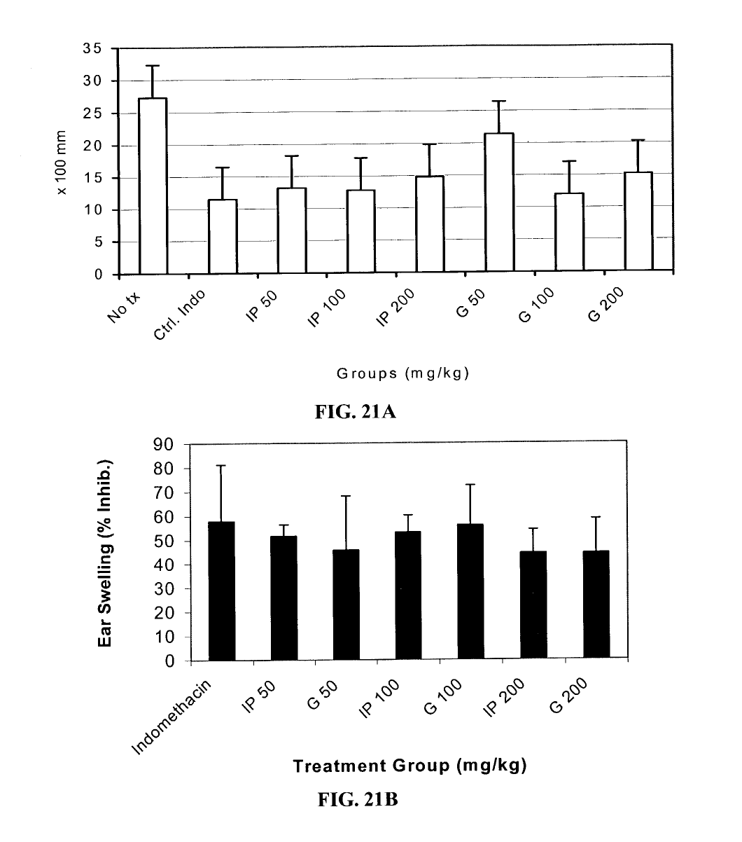

Once active botanical extracts and/or fractions and/or compounds are identified as having a novel mechanism of action and/or specific therapeutic value, chemical composition profiling and active component standardization are completed. Evaluation and confirmation of the safety and therapeutic efficacy of the compound is achieved through secondary screens with protein, cell, gene and animal models. Example 14 describes the confirmation of the anti-inflammatory activity of a standardized plant extract that was identified and developed using the Phytologix™ platform. The results are set forth in FIG. 21. The validation process was designed to establish both in vitro and in vivo efficacy, information on safety and toxicity, bio-availability and dosage. Taken collectively, the PhytoLogix™ Discovery Process establishes market identification and differentiation of the novel ingredients with a competitive advantage.

The final step of the PhytoLogix™ Discovery Process is a product development strategy directed by a bioinformatic database for intellectual property positioning, raw material sourcing and pilot scale process optimization. Pharmaceutical activity and safety/toxicology profiling are reconfirmed for the product after production to prepare for regulatory approval and to provide regulatory guidance and effective claim substantiation for customers.

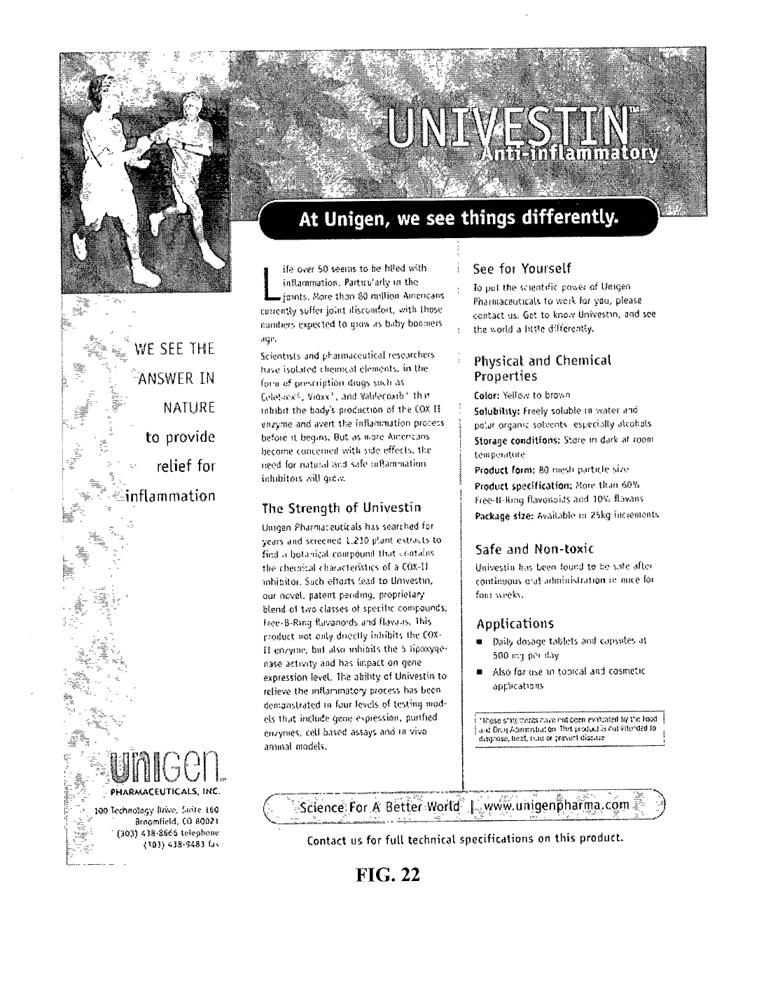

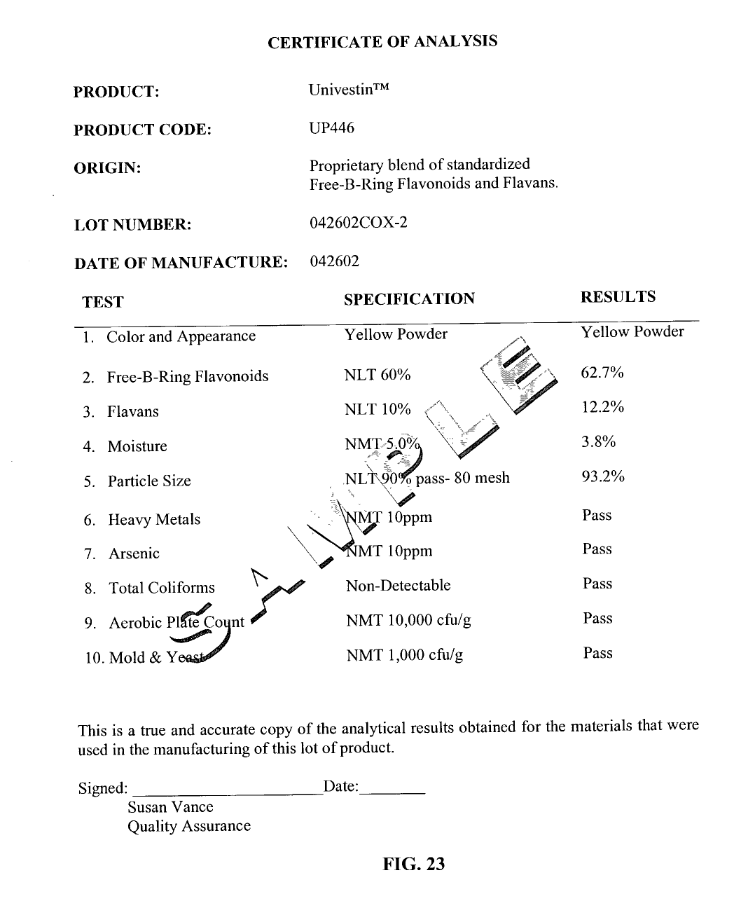

Example 15 summarizes the whole process utilizing a real life example in developing a natural COX inhibitor as a nutraceutical product. The output is a novel composition of matter referred to as Univestin™, which targets joint pain and inflammation. This composition of matter is described in U.S. patent application Ser. No. 10/104,477, filed Mar. 22, 2002, entitled “Isolation of a Dual Cox-2 and 5-Lipoxygenase Inhibitor from Acacia.”, which is incorporated herein by reference in its entirety. This product is now commercially available and FIGS. 22 and 23 set forth the selling sheet and the certificate of analysis for this product.

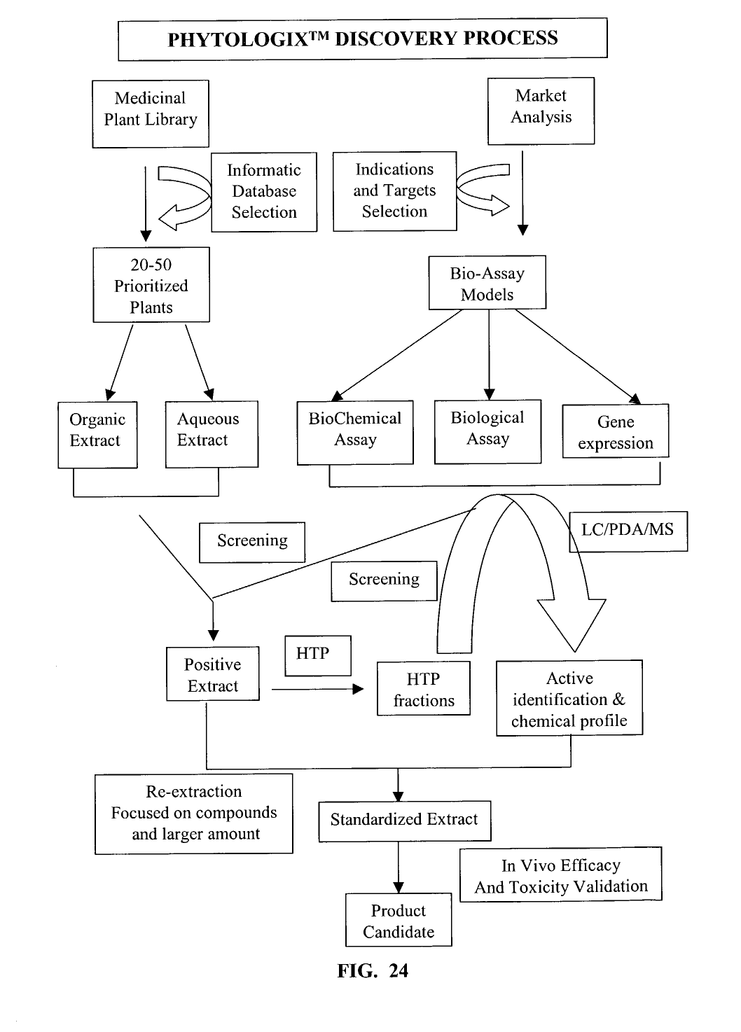

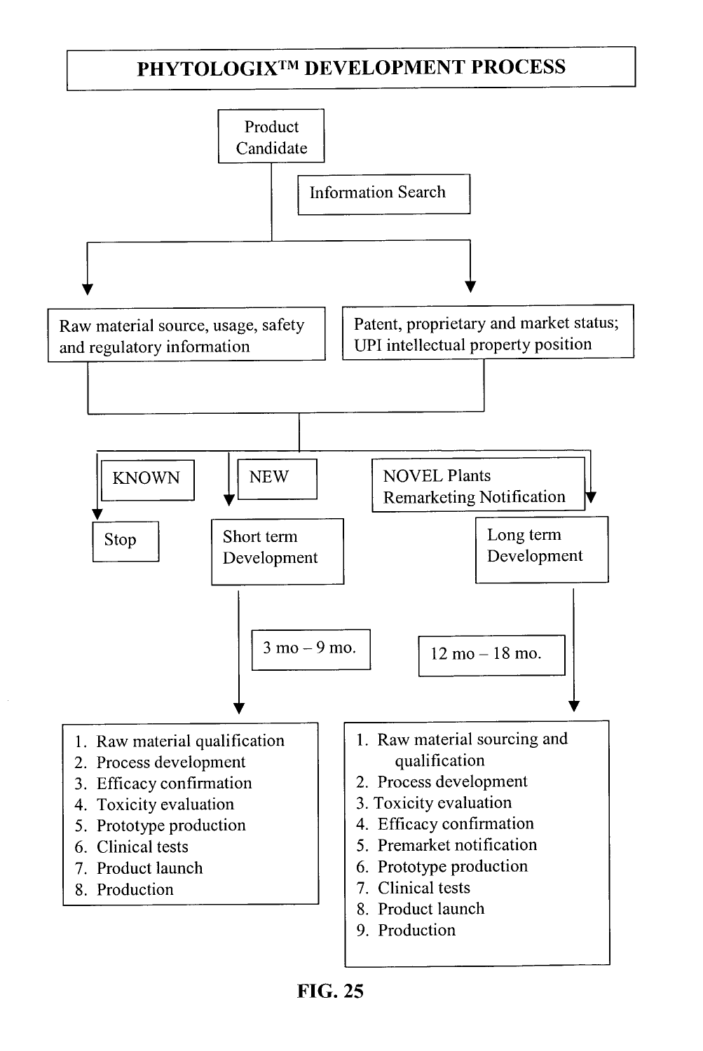

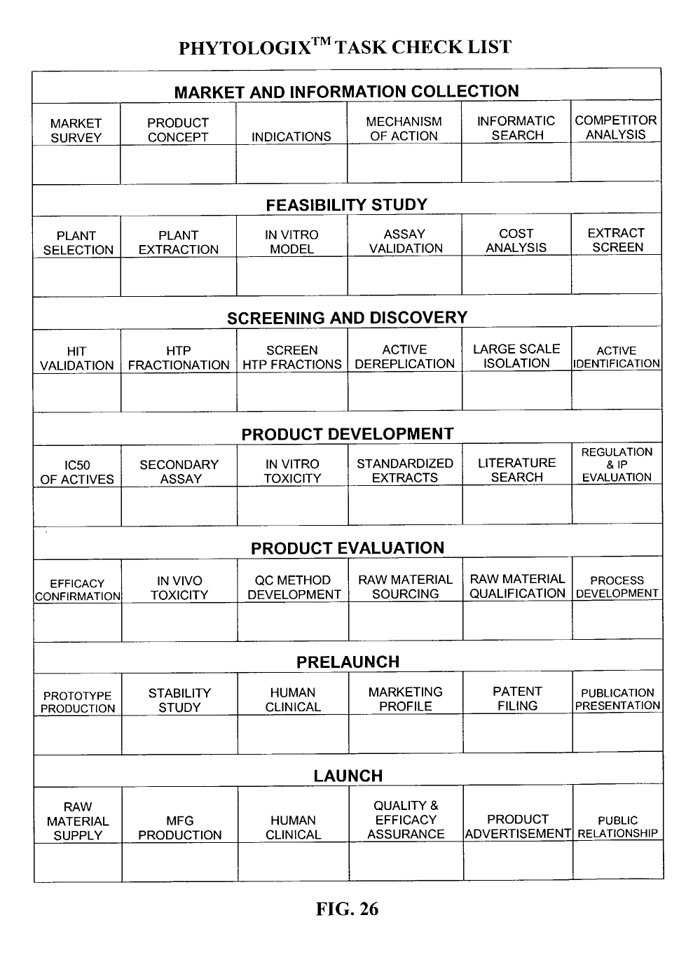

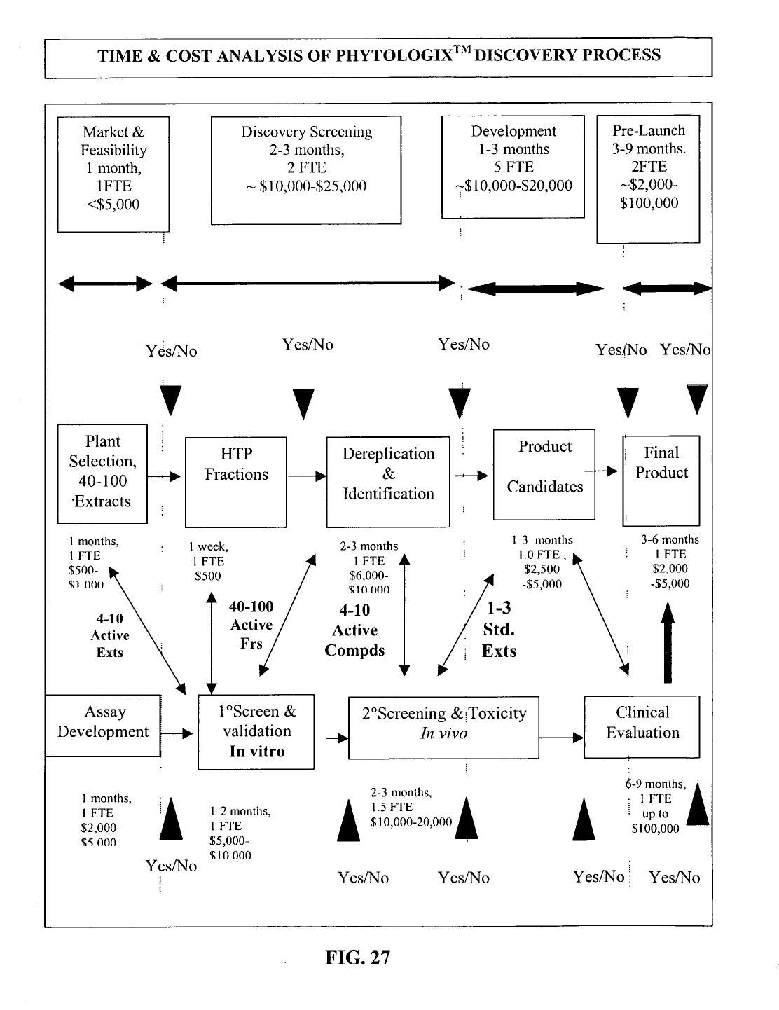

A general summary of the Phytologix™ process is provided in Example 16 and illustrated in FIGS. 24-27. FIG. 24 depicts the Phytologix™ discovery process schematically. From the analysis of plant collection libraries and market requirements, high throughput screening models were developed to assay the prioritized plant extracts. After identification of the biological activity, the pharmacological and safety profiles were generated based on a standardized extract/enriched fractions/pure compound. The output of this process is a product candidate. FIG. 25 illustrates the Phytologix™ development process schematically. With reference to FIG. 25, the product candidate, information search and product development leads to the identification of plant sources for production usage, to make recommendations on intellectual property position and market advantage. Manufacturing process development and production of pilot scale prototype product would be followed with confirmation of efficacy and safety profiles. The completion of the Phytologix™ process would be marked with successful clinical trials and final product launch. FIG. 26 illustrates a critical task checklist that may be utilized in the Phytologix™ process to keep track of the critical activities and data generations. FIG. 27 demonstrates the time, cost estimation and decision making process in the Phytologix™ process. It shows the requirement of full time employees, and the time and cost involved for each stage of discovery and development. This gives the project manager an opportunity to evaluate the progress of the project at each critical decision making point.

The following examples are provided for illustrative purposes only and are not intended to limit the scope of the invention.

### EXAMPLES

### Example 1

### Collection of Plants and Voucher Specimens

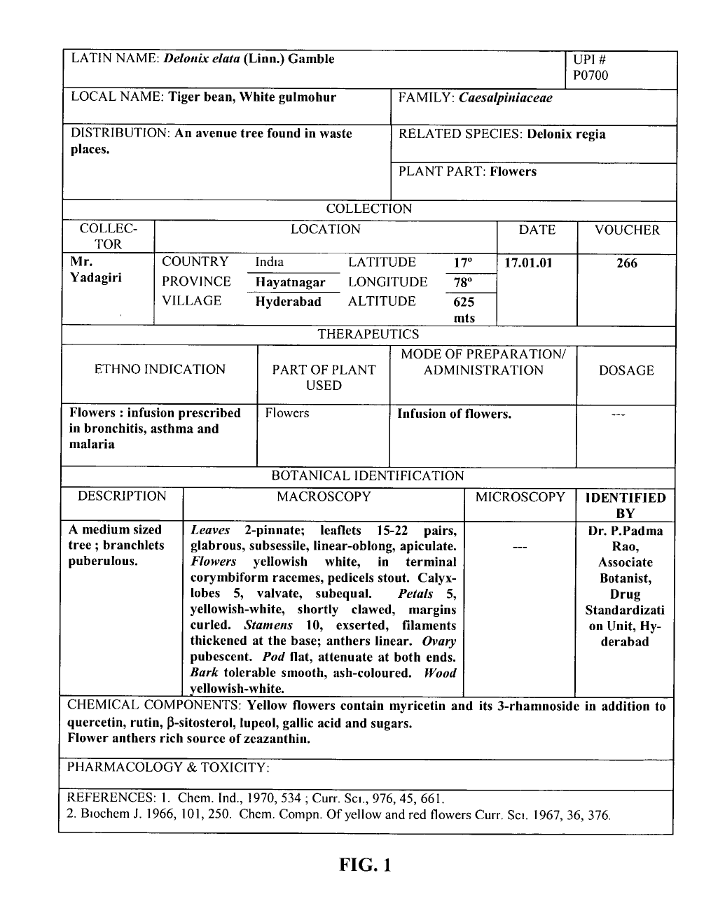

The plant to be collected was first identified and the fresh plant was then collected either from the field or from a plant farm. If applicable, the plant parts were cut from the whole plant. Enough material was collected to provide: 10-12 kg of fresh leaves, 7-8 kg of fresh fruits or seeds or whole plant or 5-6 kg of fresh stems or roots. The whole plant or plant parts (referred to hereinafter as plant/plant parts) were cleaned with water and insects, dirt and other contaminants were removed. The plant/plant parts were then dried in open air or using a mechanical dryer at a temperature lower than 60° C. The total weight of the plant/plant parts was recorded both before and after drying. Additionally, a record was kept of any changes in the plant sample that occurred as a result of the drying process. Prior to packing, the plant/plant parts were evaluated for various conditions such as, dryness, insect and fungi infection and cleanliness, etc. The plant/plant parts were then placed into a clean bag labeled with voucher number, plant name, plant parts and weight. If possible each plant sample was packed into one bag. However, if the plant sample was packed into several bags, the number of bags should also be provided on the sample label. A plant collection form (FIG. 1) was then filled out and included with the packaged plant. Several individual bags of plants were placed into a cardboard box. A packing list, including the packing date, name of the plants, voucher number, number of bags for each plant and weight of each plant was generated for each box. A desiccant bag was placed into the box and the box was sealed. A copy of collection form (FIG. 1) and packing list was sent by mail to prevent loss or damage in the process of shipping and handling.

To prepare voucher specimens, mature whole plant, including flowers and fruits were collected. The fresh plant was pressed flat and placed within newspaper or some other kind of raw paper. The paper was changed everyday until the plant was totally dry. The flowers and seeds of the plant were placed separately into small bags. If the fertile plant was not available, information about the flowers and/or fruits of the plant was obtained from the collector. An attempt was made, however, to collect the voucher specimen when the plant was flowering and fruiting. An individual voucher number was assigned to each plant. The plant voucher number, Latin name, local name, collection place, date and collector name was recorded on a label and the label was placed within the plant voucher. Two sets of vouchers were prepared, one to send to the research facility with the plant for identification purposes and one to place on file with the collector for future comparison.

Upon receipt of the plant materials by the research facility, the voucher specimen was removed and attached to the collection records or any other pertinent documents. The condition of the plant samples was checked and a plant log form was filled out for each sample. An individual number was assigned to each sample, using Pxxxx for plants, Mxxxx for marine materials, Bxxxx for bacteria and microbial, Fxxxx for fungi, Sxxxx for soils, Axxxx for animals, Ixxxx for insects, and Mxxxx for minerals, Vxxxx for vitamins, Oxxxx for organic synthetic compounds and Gxxxx for genomic modulated secondary metabolisms. This number was attached to the voucher specimen.

If the plant sample was not totally dry, it was chopped or ground into smaller pieces and freeze dried as soon as possible. A specimen (10 g, Specimen #1), was retained from each plant sample before grinding. The specimen was placed into a labeled bottle (125 mL) and stored at −20° C. prior to use. Specimen #1 was used for plant macroscopic and microscopic identification purposes only. After grinding, a specimen (100 g, Specimen #2) of the dry powder was retained and placed into a labeled bottle (250 mL) and stored at −20° C. prior to use. Specimen #2 was used for plant chemical identification and comparison. The voucher specimen, together with a copy of the collection records and a plant sample (10 g) was sent to a botanical institute for plant identification. The results of this identification were recorded on a Plant Information Form. The condition of the plant was again checked to assure that it was dry and free from infestation. The ground plant sample was then placed into a wide mouth polypropylene bottle. The material was weighed and the weight was recorded on the label. The Plant Log Form, Plant Tracking Record and Plant Information Form were then submitted to the appropriate personnel. The information from all of these forms was then input into the computer database and all forms were then appropriately filed in a secure location. As of June 2002, the Phytologix™ library contained a total of 1170 plant and other natural materials from more than 300 different families, 900 genera and more than 1100 different species. These plants were collected from China, India, Ghana, USA, and other countries in Asia, South America, North America, and Africa.

### Example 2

### Generation of Database

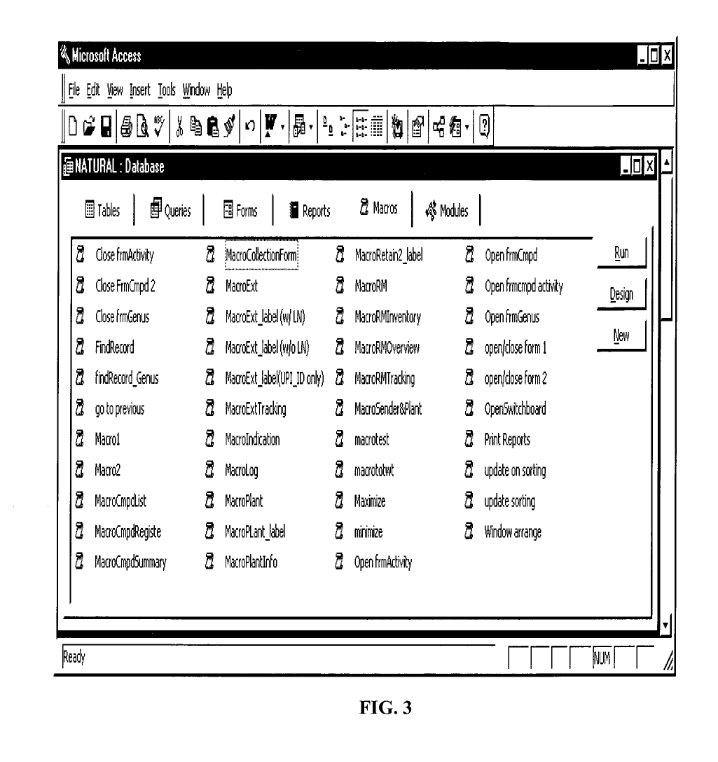

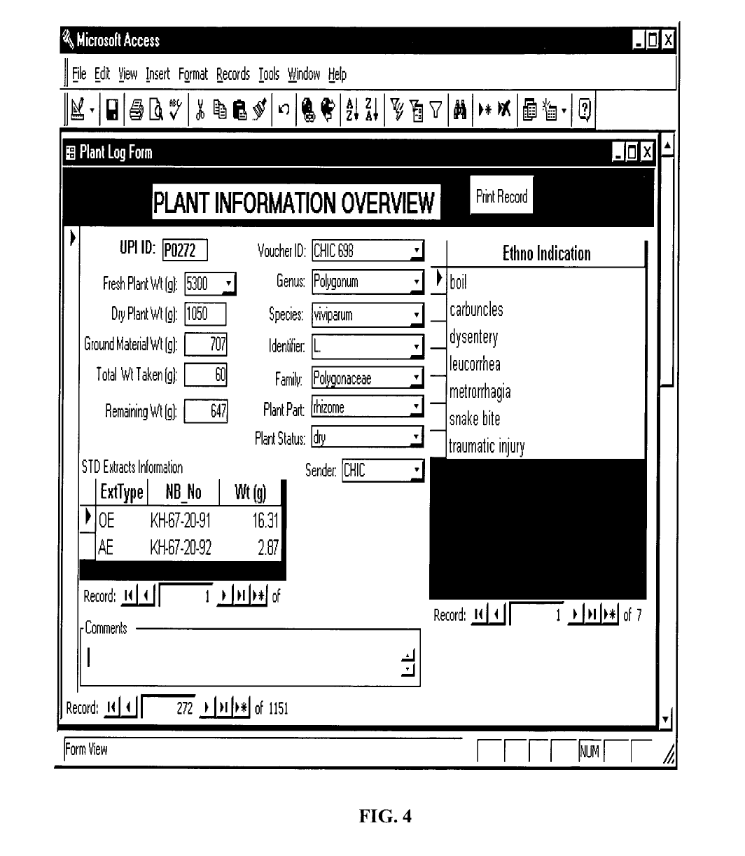

A customized Access database was developed to handle all of the information collected concerning medicinal plants and other natural materials. The database is comprised of multiple tables with specific designed relationships among those tables. As illustrated in the FIG. 2, typical tables include information such as: Log, Plant Ethno Indication, Ext., Fractionations, Ext. Tracking, Storage, Compound Type, Compound Registration, Sender, Activity, Assay, etc. Information about each sample collected, such as ID #, voucher ID, Genus, Species, Family, plant part, plant status, plant fresh weight, dry weight, geological distribution, Botanical identification, plant collection forms, extract information, ethno indication, assay results, etc. was saved in its respective table. Once entered into its respective table, the information was analyzed and searched using specifically designed macros (FIG. 3) and queries. The information was summarized in the form of reports as illustrated in FIG. 4. Table 1 sets forth the search results of medicinal plants traditionally used to treat Rheumatoid arthritis and arthritis. Such information will help to prioritize the research efforts by focusing on a limited number of plants (20-50) for a specific target. This “informatics database,” which is directed to the discovery process will significantly decrease the product discovery and development risks, costs and times and enhance the possibility of finding truly novel and efficacious products.

### Example 3

### Preparation of a Plant Extract Library

Plant material was ground to a particle size of no larger than 2 mm. Dried ground plant material (60 g) was then transferred to an Erlenmeyer flask and methanol:dichloromethane (1:1) (600 mL) was added. The mixture was shaken for one hour, filtered and the biomass was extracted again with fresh methanol:dichloromethane (1:1) (600 mL). The organic extracts were combined and evaporated under vacuum at 40° C. to provide the organic extract (see Table 2 below). After organic extraction, the biomass was air dried and extracted once with ultra pure water (600 mL). The aqueous solution was filtered and freeze-dried to provide the aqueous extract (see Table 2 below). A sample (100-200 mg) was retained from each extract (aqueous and organic) and stored at −20° C. for future reference.

Preparation of extract master plate for bioassays. A sample of each extract in the range of 70±25 mg was placed into a vial, DMSO (1.5 mL) or ultra pure water (1.5 mL) was added to each vial, and the mixture was sonicated until the solid was totally dissolved. The solution was then transferred from each vial into a well in a 96-deep well block. The position and corresponding sample code was documented. The 96-deep-well block was stored in a freezer at −70° C. prior to use. To perform the bioassays the sample was allowed to thaw and 50-200 mL of sample was used for each bioassay.

### Example 4

### Validation of the extraction methodology

Plant material was ground to a particle size of no larger than 2 mm. Dried ground plant material (60 g) was then transferred to an Erlenmeyer flask and methanol:dichloromethane (1:1) (600 mL) was added. The mixture was shaken for one hour, filtered and the biomass was extracted again with methanol:dichloromethane (1:1) (600 mL). The organic extracts were combined and evaporated under vacuum to provide the organic extract (see Table 3 below). After organic extraction, the biomass was air dried and extracted once with ultra pure water (600 mL). The aqueous solution was filtered and freeze-dried to provide the aqueous extract (see Table 3 below). After aqueous extraction, the biomass was dried and extracted twice with methanol (600 mL). The combined methanol solution was evaporated under vacuum and at 40° C. to yield the MeOH extract. The organic, aqueous and methanol extracts from same plants were analyzed with HPLC/PDA/MS for comparison of the finger print compounds. The representative results are set forth in FIGS. 5 and 6.

### Example 5

### Generation of an HTP Fraction Library from Organic Extracts

Organic extract (400 mg) was dissolved under sonication into a minimum amount of MeOH (around 1-1.5 mL) and manually loaded onto a prepacked flash column. (2 cm ID×8.2 cm, 10 g silica gel). The column was dried under vacuum until all solvent was evaporated. The column was eluted in parallel using a Hitachi high throughput purification (HTP) system with an unique gradient mobile phase of (A) 50:50 EtOAc:hexane and (B) methanol from 100% A to 100% B in 30 minutes at a flow rate of 5 mL/min. The separation was monitored using a broadband wavelength UV detector and the eluents were collected in 88 fractions in a 96-deep-well plate at 1.9 mL per well using a Gilson fraction collector. The sample plate was dried under low vacuum and centrifugation with SpeedVac Plus from Savant (model #SC250DDA). FIGS. 7A and 7B illustrate the analysis of the HTP fractions using thin layer chromatography (TLC). This figure demonstrates that HTP yielded impressive separation of different types of compounds based on polarity. The separated components may be distributed in 6-10 cells and in most cases each cell contained either a single compound or at most less than three compounds.

FIG. 8 depicts the weight distribution of the sample in each well. There were several peaks that matched each other in the weight distribution profile against the TLC compound spots. DMSO (1.5 mL) was added to each well to dissolve the samples and the 96-deep-well plates were stored at −70° C. The master fraction plates were thawed at room temperature and a portion of each solution (50-200 μL) was taken from each well to make a daughter plate for any designated bioassays. It took approximately 40 minutes to complete two HTP column fractionations and approximately 5 hours to dry eight 96-deep-well plates. Daily throughput for organic extracts is 14 columns and 1232 fractions. Table 4 depicts the cost analysis of the high throughput fractionation of the organic extracts.

### Example 6

### Generation of an HTP Fraction Library from Aqueous Extracts

Aqueous extract (750 mg) was dissolved in deionized (DI) water (5 mL), filtered through a 1 μm syringe filter and transferred to a 4 mL HPLC vial. The solution was then injected by an autosampler onto a prepacked reverse phase column. (C-18, 15 μm particle size, 2.5 cm ID×10 cm with precolumn insert). The column was eluted using a Hitachi high throughput purification (HTP) system with a gradient mobile phase of (A) water and (B) methanol from 100% A to 100% B in 20 minutes, followed by 100% methanol for 5 minutes at a flow rate of 10 mL/min. The separation was monitored using a broadband wavelength UV detector and the eluent was collected in 88 fractions in a 96-deep-well plate at 1.9 mL/well using a Gilson fraction collector. The methanol was removed under low vacuum and centrifugation with a SpeedVac Plus from Savant (model #SC250DDA) and the plate was freeze-dried. Ultra pure water (1.5 mL), which was sterile filtered and Endotoxin tested, was added to each well to dissolve the samples and the 96-deep-well plate was stored at −70 ° C. prior to use. The master fraction plates were thawed at room temperature and a portion (50-200 μL) of solution was taken from each well to make a daughter plate for any designated bioassays. FIGS. 9A-9L illustrate the reproducibility of the HTP separation of an aqueous extract from whole plant of Ainsliaea henryi. The aqueous extracts were separated three times on 4 parallel C-18 columns on the HTP and total of twelve 96-deep well plates were generated. The HTP/UV chromatograms from 12 column separations were identical and the samples were combined based on the same well position from the twelve plates.

It takes approximately 20 minutes to complete two HTP column fractionations and approximately 10 hours to dry eight 96-deep-well plates. Daily throughput for aqueous extracts is 32 columns and 2618 fractions. Table 5 depicts the cost analysis of the high throughput fractionation of the aqueous extracts.

### Example 7

### Screening of the Plant Extract Library for Natural Inhibitors of COX-2 and COX-1

The bioassay directed screening process for the identification of specific COX-2 inhibitors was designed to assay the peroxidase activity of the enzyme as described below. In order to screen for compounds that inhibited the activity of COX-1 and COX-2, a high throughput, in vitro assay was developed that utilized the inhibition of the peroxidase activity of both enzymes (Raz and Needleman et al. (1990) J. Biol. Chem. 269:603-607). Briefly, a known concentration of Univestin™ and/or its individual ingredients—Free-B-ring flavanoids or flavans was titrated against a fixed amount of the COX-1 and COX-2 enzymes, respectively. A cleavable, peroxide chromophore was included in the assay to visualize the peroxidase activity of each enzyme in the presence of arachidonic acid as a cofactor. Typically, assays were performed in a 96-well format. Each inhibitor, taken from a 10 mg/mL stock in 100% DMSO, was tested in triplicate at room temperature using the following range of concentrations: 0, 0.1, 1, 5, 10, 20, 50, 100, and 500 μg/mL. To each well, 150 μL of 100 mM Tris-HCl, pH 7.5 was added, together with 10 μL of 22 μM Hematin diluted with tris buffer, 10 μL of inhibitor diluted with DMSO, and 25 units of either the COX-1 or COX-2 enzyme. The components were mixed for 10 seconds on a rotating platform, after which 20 μL of 2 mM TMPD and 20 μL of 1.1 mM arachidonic acid was added to initiate the reaction. The plate was shaken for 10 seconds and then incubated for 5 minutes before reading the absorbance at 570 nm. Luminescence was read using a Wallac Victor 2 plate reader. The inhibitor concentration vs. % inhibition was plotted and the IC₅₀ determined by taking the half-maximal point along the isotherm and intersecting the concentration on the x-axis. The IC₅₀ was then normalized to the number of enzyme units in the assay. FIG. 10 shows the positive hit rate resulting from the screening of 1230 plant extracts. The inhibition of COX-2 peroxidase by extracts from representative plant species is set forth in Table 6. The data in Table 6 is presented as the percent of peroxidase activity relative to the recombinant ovine COX-2 enzyme and substrate alone. The percent inhibition by the representative organic extracts ranged from 30% to 90%.

Comparison of the relative inhibition of the COX-1 and COX-2 isoforms requires the generation of IC₅₀ values for each of these enzymes. The IC₅₀ is defined as the concentration at which 50% inhibition of enzyme activity in relation to the control is achieved by a particular inhibitor. In the instant case, IC₅₀ values were found to range from 6 to 50 μg/mL and 7 to 80 μg/mL for the COX-2 and COX-1 enzymes, respectively, as set forth in Table 7 . Comparison, of the IC₅₀ values of COX-2 and COX-1 demonstrates the specificity of the organic extracts from various plants species for each of these enzymes. The organic extract of Scutellaria lateriflora for example, shows preferential inhibition of COX-2 over COX-1 with IC₅₀ values of 30 and 80 μg/mL, respectively. While some extracts demonstrate preferential inhibition of COX-2, others do not. Examination of the HTP fractions and the purified compounds isolated from these fractions is necessary to determine the true specificity of inhibition for these extracts and compounds.

### Example 8

### Screening of the Plant Extract Library for Natural Inhibitors Tyrosinase

Tyrosinase activity was determined using a modified method of Pomerantz (Pomerantz (1991) J Biol. Chem. 241:161-8). Briefly, crude extracts were dissolved in DMSO at a concentration of 30 mg/mL. Samples were then diluted 1:10 in potassium phosphate buffer pH 6.8. Further dilutions were performed in 10% DMSO/buffer. For large-scale screening, the assay was converted to a 96 well format. Sample test wells consisted of 50 μL buffer, 50 μL of 0.5 mg/mL extract, 50 μL of 2 mM L-dopa and 50 μL of 50 U/mL mushroom tyrosinase. Positive control consisted of the above, except sample was replaced with 10% DMSO/buffer. The substrate was added last, with a 12 channel multi-well pipette to initiate the reaction. The plate was read immediately in a 96 well plate reader at 450 nm to detect the formation of dopachrome. The plate was then incubated at room temperature and read again exactly one minute later. The change in absorbance was linear for 2 minutes. Control rate was determined to be optimal at Δ0.2 A/min. at 450 nm. The percent inhibition for test samples was calculated using the following formula:

Percent Inhibition=(Rc-Rs)/Rc×100

Rc: Δ absorbance/minute at 450 nm without sample (control)

Rs: Δ absorbance/minute at 450 nm with sample

Crude organic and aqueous plant extracts were tested against purified mushroom tyrosinase in the 96 well plate format. The concentrations of L-Dopa substrate and tyrosinase enzyme were scaled down linearly using a modified method of Pomerantz. FIG. 11 depicts the tyrosinase inhibition results of 396 organic extracts derived from various plant species. Of 774 plant extracts, there were 43 extracts which showed >60% inhibition (5.6% positive hits); 6 plants were identified with active fractions that have an IC₅₀<100 μg/mL (0.78% confirmed hits); and 6 active compounds were isolated and identified.

### Example 9

### Screening HTP Fraction Library for Inhibitors of COX Peroxidase

Individual bioactive organic and aqueous extracts from Example 7, were further characterized by examining each of the HTP fractions for the ability to inhibit the peroxidase activity of both the COX-1 and COX-2 recombinant enzymes using the method described in Example 7. FIG. 12 depicts the broad wavelength UV chromatogram of HTP fractions of the aqueous extract of Camellia sinensis (P0605). The representative COX inhibitory results are depicted in FIG. 13, which demonstrates the inhibition of COX-2 and COX-1 activity by HTP fractions from Camellia sinensis (P0605), generated as described in Examples 3 and 6. The profile depicted in FIG. 13 shows a peak of inhibition that is located between fractions C4 to E4 in a total of 16 fractions with certain level of selectivity for COX-1. Both the COX-1 and COX-2 enzymes demonstrate multiple peaks of inhibition suggesting that there is more than one compound contributing to the initial inhibition profiles of the aqueous extract from Camellia sinensis (P0605).

### Example 10

### Generation of a Database Comprised of Structures and Spectroscopic Characteristics

A database comprised of 250 pure natural products with representative structure types in a quantity of 5-500 mg and a purity of >90% (HPLC) was generated by internal isolation of the compounds and by purchasing the compounds from commercial sources, such as Sigma, Indofine, and Chromadex. Each compound was dissolved in methanol (1 mg/mL). Further dilution and concentration may be necessary for individual compounds due to different UV absorption and mass ionization properties. The sample solutions were analyzed by HPLC using a Luna C18 column (2×50 mm, 3 μm) at a flow rate of 0.4 mL/min and a temperature of 35° C. The column was eluted in 8.5 minutes with a gradient system of 10% to 90% acetonitrile (ACN) in water from 0-4 minutes, 100% ACN from 4.1 to 6.0 minutes and equilibrated between 6.1 to 8.5 minutes with 10% ACN in water. The eluent was analyzed with a Photo Diode Array detector with wavelength from 200-500 nm; and ion trap MS under the following conditions: detector 475 v, focus 35 v, drift 40 v, SSI chamber 0.5 kv, aperture 1150° C., aperture 2120° C., cover plate 250° C. and negative or positive detection. The retention time, UV spectrum, molecular ion and fragmentations were recorded and saved in a searchable library. Table 8 sets forth the typical information included in the Structures and Spectroscopic database. In the dereplication process, unknown fractions were analyzed under the same conditions and the HPLC peaks from PDA detection were searched in the UV library for structural skeleton and compound type. The molecular ion and retention time were used to identify known compounds by searching the Dictionary of Natural Products and the NERAC database.

### Example 11

### Dereplication of the HTP Fraction Library for Inhibitors of COX Peroxidase

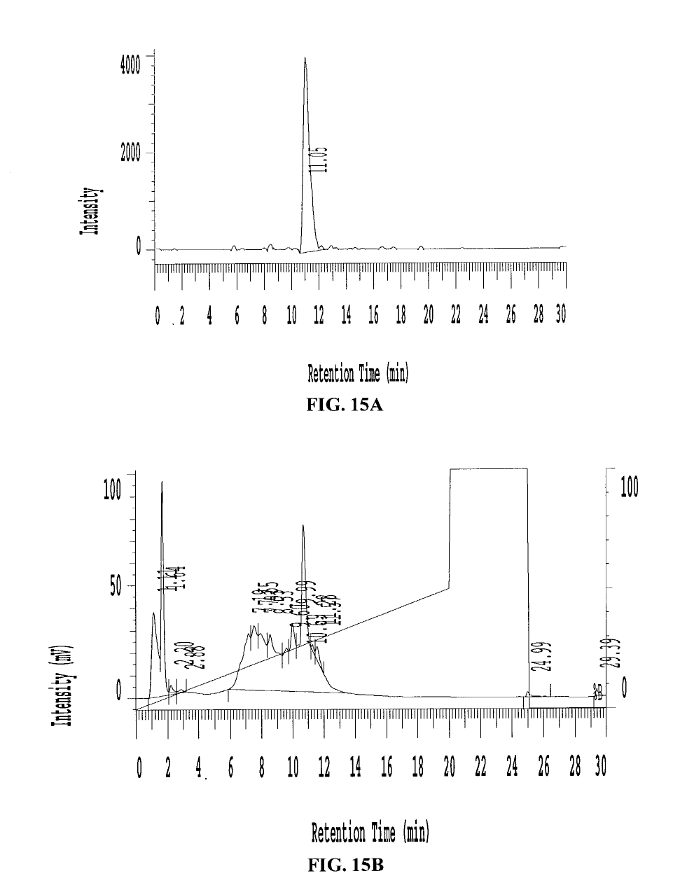

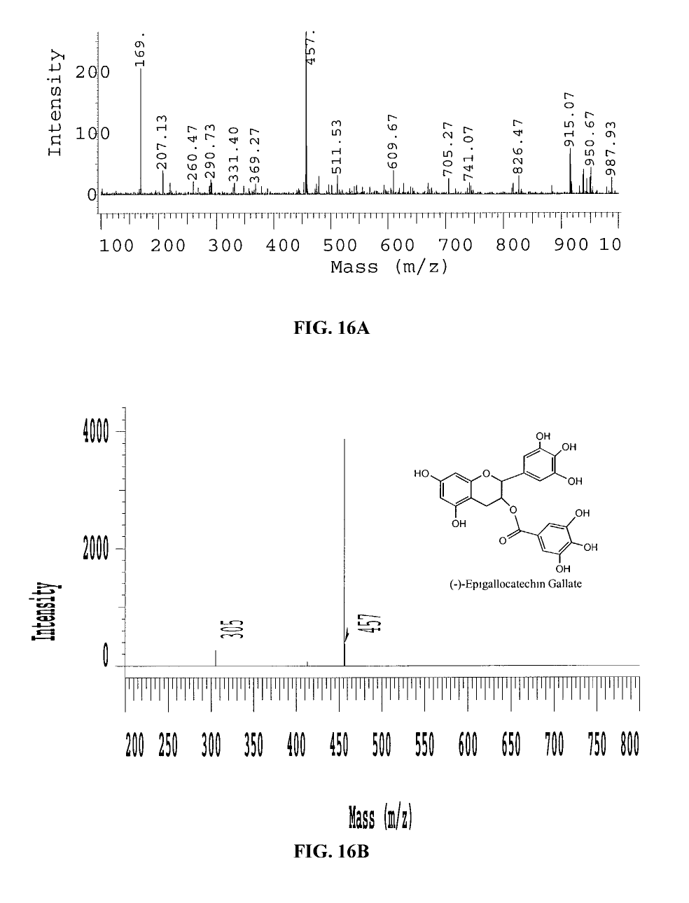

The dereplication process was initiated after the bioassay results from the HTP plates were obtained. The results are set forth in FIG. 13, which shows that the COX inhibitory activity resided in fraction C4 to fraction E4 in a total of 16 fractions. Those fractions were analyzed individually on LC/PDA/MS as described in Example 10. The results are illustrated in FIG. 14, which depicts the online PDA/MS Base Ion Chromatogram (BIC) of bioactive fraction D3. FIGS. 15A and 15B depict the UV and MS chromatogram of fraction D3 analyzed off-line after the fraction was collected. The molecular ion spectra for fraction D3 were identical regardless of whether the analysis was performed online or off-line, as shown in the FIG. 16A. Based upon a search of the Structure and Spectroscopic library using the experimental data and information obtained from the Dictionary of Natural Products, fraction D3 contained one major known compound Epigallocatechin Gallate (EGCG) (FIG. 16B), which is a well known COX inhibitor. Using the same strategy, all 16 active HTP fractions were dereplicated and found to contain known catechins and flavonoids as illustrated in the FIG. 17.

### Example 12

### Dereplication of the HTP Fraction Library for Inhibitors of Tyrosinase

Inhibition of melanogenesis was determined by a modified method of Siegrist and Eberle (x). B16 F1 mouse melanoma cells (2.0×10⁴ cells/mL) were subcultured in GibcoBRL Modified Eagle Medium (10% FBS, 1% Gibco non-essential amino acids, 1% PSG, 1.5% Gibco vitamin solution). After 3 days incubation (37° C., 5% CO₂) cells were seeded (2500 cells/well, 200 μL) in 96 well sterile culture plates (Costar) and incubated overnight (37° C., 5% CO₂). The next day, cell culture medium was replaced with 100 μL fresh medium. Extract samples were dissolved in DMSO at a concentration of 30 mg/mL and diluted 1:1000 in cell culture medium on separate, sterile, 96 well plates. Samples (50 μL) were transferred from dilution plates to cell culture plates using a 12 well multi-well pipette. α-Melanocyte stimulating hormone (α-MSH) (Sigma) was added to all positive wells (150 pM, 50 μL) to stimulate melanogenesis. Sample wells containing no α-MSH were used to determine sample absorbance at 450 nm unrelated to melanin pigment formation as control. Melanin pigment formation was visible after four days. The degree of melanin formation was determined at 450 nm in a 96 well plate reader. Percent inhibition of samples was determined by the formula:

Percent Inhibition=[(Ac MSH+)−(Ac MSH−)]/[(As MSH+)−(Ac MSH−)][Ac MSH+)−{As MSH−)]×100

Ac MSH+: Absorbance at 450 nm of cells containing no sample, with MSH

Ac MSH−: Absorbance at 450 nm of cells containing no sample, without MSH

As MSH+: Absorbance at 450 nm of cells containing sample, with MSH

As MSH−: Absorbance at 450 nm of cells containing sample, without MSH

The active organic extract isolated from Mallotus repandus (whole plant) (P0368) was fractionated with HTP as described in the Example 5. All of the HTP fractions were tested for tyrosinase inhibitory activity versus cell toxicity and the results are set forth in FIG. 18. As can be seen in FIG. 18 there are multiple of peaks, indicating the presence of a number of active components in the crude extract. The largest activity peaks, located from fraction C10 to D10, may be due to cell toxicity rather than enzyme inhibition. The most interesting activity resided at the peak between fractions D2 to D7, which had no cell toxicity. The HPLC/PDA/MS analysis of those fractions (FIGS. 19A-F) showed an increase in negative ion intensity at a retention time 16.33 minutes, which is superimposed with the position of the tyrosinase inhibitory activity. Further analysis of this peak by Ultra Violet and Mass Spectrometry (FIG. 19G), indicated that the skeleton of this compound was that of a gallate. Based upon a search of the Structure and Spectroscopic library using the experimental data and information obtained from the Dictionary of Natural Products, it was determined that this peak corresponds to the known polyphenol, Pterocaryanin B. The structure of this compound is set forth in FIG. 19H. Polyphenols are well known as having tyrosinase inhibitory activity. Thus, the dereplication process quickly identified that the positive hit from crude extract and HTP fractions was due mainly to a known compound in addition to some cell toxicity from known sources. There was no need to further pursue this plant extract.

### Example 13

### Isolation and Purification of the Active Free-B-Ring Flavonoids from the Organic Extract of Scutellaria orthocalyx

The organic extract (5 g) from the roots of Scutellaria orthocalyx, isolated as described in Example 3, was loaded onto a prepacked flash column (120 g silica, particle size 32-60 μm, 25 cm×4 cm) and eluted with a gradient mobile phase of (A) 50:50 EtOAc:hexane and (B) methanol from 100% A to 100% B in 60 minutes at a flow rate of 15 mL/min. The fractions were collected in test tubes at 10 mL/fraction. The solvent was evaporated under vacuum and the sample in each fraction was dissolved in 1 mL of DMSO and an aliquot of 20 μL was transferred to a 96 well shallow dish plate and tested for COX inhibitory activity. Based on the COX assay results, active fractions #31 to #39 were combined and evaporated. Analysis by HPLC/PDA and LC/MS showed a major compound with a retention time of 8.9 minutes and a MS peak at 272 m/z. The product was further purified on a C18 semi-preparation column (25 cm×1 cm), with a gradient mobile phase of (A) water and (B) methanol, over a period of 45 minutes at a flow rate of 5 mL/minute. Eighty eight fractions were collected to yield 5.6 mg of white solid. Purity was determined by HPLC/PDA and LC/MS, and comparison with standards and NMR data. ¹H NMR: δppm. (DMSO-d6) 8.088 (2H, m, H-3′, 5′), 7.577 (3H, m, H-2′, 4′, 6′), 6.932 (1H, s, H-8), 6.613 (1H, s, H-3). MS: [M+1]+=271 m/e. The compound was identified as Baicalein. The IC₅₀ for COX-1 was 0.18 μg/mL/unit of enzyme while the IC₅₀ for COX-2 was 0.28 μg/mL/unit (FIG. 20).

### Example 14

### In vivo Study of COX Inhibitory Activity of a Standardized Nutraceutical Extract

In vivo inhibition of inflammation was measured using two model systems. The first system (ear swelling model) measures inflammation induced directly by arachidonic acid. This is an excellent measure of COX-2 inhibition, but does not measure any of the cellular events which would occur upstream of arachidonic acid liberation from the cell membrane phospholipids by phospholipase A2 (PLA2). Therefore, to determine how inhibitors function in a more biologically relevant response the air pouch model was employed. This model utilizes a strong activator of complement to induce an inflammatory response that is characterized by a strong cellular infiltrate and inflammatory mediator production including cytokines as well as arachidonic acid metabolites.

The ear swelling model is a direct measure of the inhibition of arachidonic acid metabolism as previously described (Greenspan et al. (1999) J. Med. Chem. 42:164-172; Young et al. (1984) J. Invest. Dermat. 82:367-371). Arachidonic acid in acetone is applied topically to the ears of mice. The metabolism of arachidonic acid results in the production of proinflammatory mediators produced by the action of enzymes such as COX-2. Inhibition of the swelling is a direct measure of the inhibition of the enzymes involved in this pathway. Seven groups of 5 Balb/C mice were given three dosages of test compounds either interperitoneally (I.P.) or orally by gavage, 24 hours and 1 hour prior to the application of arachidonic acid (AA). AA in acetone (2 mg/15 μL) was applied to the left ear, and acetone (15 μL) as a negative control was applied to the right ear. After 1 hour the animals were sacrificed by CO₂ inhalation and the thickness of the ears was measured using an engineer's micrometer. Controls included animals given AA, but not treated with anti-inflammatory agents, and animals treated with AA and indomethacin (I.P.) at 5 mg/kg.

The results are set forth in FIG. 21, which shows the effects of three extracts delivered either orally by gavage or interperitoneally (IP) at two time points (24 hours and 1 hour). Free-B-Ring Flavonoids isolated from S. baicalensis inhibited swelling when delivered by both IP and gavage although more efficacious by IP. (FIGS. 21A and 21B). Free-B-Ring Flavonoids isolated from S. orthocalyx inhibited the generation of these metabolites when given IP, but not orally, whereas extracts isolated from S. lateriflora, while being efficacious in vitro, had no effect in vivo (data not shown).

### Example 15

### Development of a Natural COX-2 Inhibitor as a Nutraceutical Product as Result of the PhytoLoix™ Platform

The PhytoLogix™ process has been used for years to screen thousands of plant extracts in order to find novel nutraceutical ingredients containing the chemical characteristics of COX-II inhibitors. A library of 1230 plant extracts was screened against multiple enzymatic and cell type assays for natural COX-2 inhibitors with 1.8% positive hits. The 22 active extracts were further examined using the high throughput purification system described above and the isolated pure compounds were tested using the COX assays described above. The biological activities of the pure compounds and plant extracts were confirmed with ovine COX-1 and COX-2 enzymes, human COX-2 enzyme, bee venom PLA2, Human 5-LO, human peripheral blood cells, and THP-1 cell line assays. Those extracts that were determined to be efficacious based on in vitro models, were then tested for the ability to inhibit inflammation in vivo using a both air pouch and topical ear-swelling models when administered by multiple routes (IP and oral). To date, these studies have resulted in the identification of Free-B-Ring flavonoids and flavans as anti-inflammatory agents, with activities through all levels of testing.

These extensive efforts have lead to the discovery of a novel composition of matter, referred to as Univestin™, which is described in U.S. patent application Ser. No. 10/104,477, filed Mar. 22, 2002, entitled “Isolation of a Dual Cox-2 and 5-Lipoxygenase Inhibitor from Acacia.” This composition of matter is comprised of a blend of two classes of specific compounds, Free-B-Ring Flavonoids and flavans. This composition of matter not only directly inhibits the COX-2 enzyme, but also inhibits 5-lipoxygenase activity and has demonstrated to have an impact at the gene expression level. The ability of Univestin™ to inhibit the inflammatory process has been demonstrated in four levels of testing models that include gene expression, purified enzymes, cell based assays and in vivo animal models. The efficacy of this product has been evaluated against pharmaceutical drugs and other standardized plant extracts. With respect to inhibition of COX-2, in general, Univestin™ performs 8-10 times better than ibuprofen and is equivalent or better in vivo than indomethacin, a potent anti-inflammatory available by prescription only. Additionally, Univestin™ has advantages over these two drugs in that it also inhibits the production of LTB4 in cells undergoing an inflammation response, whereas ibuprofen and indomethacin may only inhibit release from cells. It is believed that this is the first report of a correlation between Free-B-Ring flavonoids and COX-2 inhibitory activity. It is also believed that this is the first report of flavans inhibiting the 5-LO pathway. This novel blending of two specific classes of compounds for the prevention and treatment of COX-2 and 5-LO mediated diseases and conditions, represents a new class of nutraceuticals for the treatment of several inflammatory diseases. The product and its ingredients have been evaluated for safety on cell and animal models. In the acute protocol, an individually standardized extract containing a high concentration of Free-B-Ring flavonoids and flavans, as well as, the product Univestin™ given at a dosage of 2 grams/kg (20 times over the human daily dose of 500 mg) produced no abnormalities in weight gain, appearance, behavior, gross necropsy appearance of organs, histology of stomach and liver and blood work.

FIG. 22 depicts an example of the selling sheet of the nutraceutical product—Univestin™ and FIG. 23 is the Certificate Of Analysis (COA) for the product.

### Example 16

### Phytologix™ Process for the Discovery of Nutraceutical and Cosmetic Products

The Phytologix™ process for the discovery of novel nutraceutical and cosmetic compositions can be illustrated in two separate protocols as set forth schematically in FIGS. 24 and 25. As shown in FIG. 24, the PhytoLogix discovery starts with a collection of thousand medicinal plants stored in a Medicinal Plant Library. A search of the informatic database based on the indications and usages would likely yield 20 to 50 medicinal plants with similar traditional applications. Those plants would then be extracted as described in Example 3 and the organic and aqueous extracts screened against biochemical, biological and gene expression targets that have been developed, preferable in high throughput models, based on the selected targets and indications as described above. If possible, the whole plant library, in the form of extracts and/or HTP fractions, could be screened through the high throughput screening (HTS) system to maximize the potential number of hits. The positive hits would then be subjected to fractionation, dereplication, isolation and re-assay, as described above to enable the identification of the novel active natural products, as illustrated in the above examples. Standardization of the plant extracts and/or enrichment and/or purification would then continue on the basis of the activity profile and chemical fingerprints. Secondary efficacy assays and evaluation of safety and toxicity of the standardized extracts and/or enriched ingredients and/or the pure active compounds on in vitro and in vivo models would optimize the multiple potentials to a limited number of product candidates.

The PhytoLogix™ process, as illustrated in the FIG. 25, begins with product candidates whose pharmacological, chemical and safety profiles have been created from previous discovery processes. The further search for information on the candidates is focused on intellectual position, original plant sourcing for potential production, market and regulations. These efforts will lead to a conclusion about the novelty of the products, market potential and further development plan. The last phase of development will generate a manufacturing process, quality control methodology, prototype product, further confirmation of efficacy and safety based on the prototype products, clinical evaluation and final product launch.

To make the Phytologx™ process time efficient and cost sensitive, including practical guidelines to direct the product discovery and development efforts, a couple of process control mechanisms have been developed. As shown in the FIG. 26, a Phytologix™ task checklist, to be used during the different stages of the discovery and development process, would be helpful to carry out the critical tasks and avoid missing important information and data on the final products: Preparation of a cost and time estimation, as illustrated in the FIG. 27 would provide the project manage with a general outline of the labor, budget and time requirements of the whole process. The most critical part of the analysis is the decision making process based on the progress of the project and the conclusions derived from critical data points.

TABLE 1
Search Results of Medicinal Plants Traditionally Used to Treat
Rheumatoid Arthritis and Arthritis

| Rheumatoid arthritis | | Arthritis | |
|---|---|---|---|
| P0110 | Uvaria microcarpa | P0126 | Wikstroemia micrantha |
| P0177 | Clerodendrum bungei | P0193 | Cajanus |
| P0340 | Zanthoxylum frazineum | P0236 | Anemone tomentosa |
| P0369 | Ampelopsis delavayana | P0239 | Livistona chinensis |
| P0412 | Peucedanum dielsianum | P0397 | Brassica juncea |
| P0414 | Lycopodium japonicum | P0536 | Lepidium apetalum |
| P0437 | Sargentodoxa cuneata | P0574 | Imperata cylindrica |
| | | | var. major C.E. |
| P0444 | Drynaria baronii | P0582 | Ligusticum brachylobum |
| P0449 | Aconitum carmichaeli | P0588 | Pharbitis nil |

TABLE 2
Representative Organic and Aqueous Extracts from various plant species

| | | | | Organic | Aqueous |
|---|---|---|---|---|---|
| Plant Name (Latin) | Plant Part | ID # | Amount | Extract | Extract |
| Catharanthus Roseus (White) | Whole plants | P0066 | 60 g | 5.16 g | 5.49 g |
| Scutellaria baicaensis | Roots | P0987 | 60 g | 9.18 g | 7.18 g |
| Cassia tora | Seeds | P0124 | 60 g | 10.67 g | 7.7 g |
| Mahonia fortunei | Stems | P0585 | 60 g | 4.17 g | 2.26 g |
| Caesalpiniaceae Afzelia | Leaves | P0079 | 60 g | 3.21 g | 4.58 g |
| Gardenia jasminoides | Fruit | P0012 | 60 g | 8.4 g | 9.64 g |
| Albizzia julibrissin | Bark | P0430 | 60 g | 5.87 g | 2.56 g |
| Magnolia biondii | Flowers | P0451 | 60 g | 5.91 g | 4.17 g |
| Angiopteris Omeiensis | Rhizomes | P0095 | 60 g | 4.8 g | 6.78 g |

TABLE 3
Comparison of Organic, Aqueous and Methanol Extracts from Different
Plant Materials

| | | | Organic | Aqueous | MeOH |
|---|---|---|---|---|---|
| | | | Extract | Extract | Extract |
| ID # | Latin Name | Plant part | (g) | (g) | (g) |
| P0490 | Daphne genkwa Sieb. Et Zucc. | flower | 6.178 | 5.022 | 1.289 |
| P0491 | Magnolia officinalis Rehd. Et Wiis | trunk bark | 7.617 | 2.44 | 0.485 |
| P0492 | Portulaca oleracea L. | whole plant | 2.579 | 6.881 | 0.577 |
| P0493 | Thalictrum glandubsissimum | rhizome | 5.356 | 4.919 | 0.895 |
| P0495 | Crataegus Pinnatifida Bge. | fruit | 14.243 | 8.56 | 0.39 |
| P0496 | Perilla Frotescans (L.) Britt | leaf | 3.614 | 5.197 | 0.919 |

TABLE 4
Cost Analysis of High Throughput Fractionation of Organic Extracts

| | supply | material | material | | total | total |
|---|---|---|---|---|---|---|
| Items | (1 smp) | price | unit price | cost/smp | cost/smp | cost/fraction |
| MeOH | 105 mL | $54/20 L | $0.0027/mL | $0.28 | $15.32 | $0.16 |
| EtOAc | 40 mL | $174.05/20 L | $0.0087/mL | $0.35 | | |
| Hexane | 40 mL | $116.88/16 L | $0.0073/mL | $0.29 | | |
| column | | $137/20 | $6.85 | $6.85 | | |
| deep well | | $200/50 | $4/ea | $4.00 | | |
| well mat | | $150/50 | $3/ea | $3.00 | | |
| scintilation | | $128.55/500 | $0.26/ca | $0.26 | | |
| vial (20 mL) | | | | | | |
| syringe | | $28.64/100 | $0.29/ca | $0.29 | | |

Total cost: $15.32/sample
$0.16/fracton

TABLE 5
Cost Analysis of High Throughput Fractionation of Aqueous Extracts

| | | | | | | total |
|---|---|---|---|---|---|---|
| | Supply | material | material | | total | cost per |
| Items | (2 smps) | price | unit price | cost/smp | cost/smp | fraction |
| MeOH | 550 mL | $54/20 L | $0.0027/mL | $0.75 | $10.94 | $0.11 |
| THF | 100 mL | $54.70/4 L | $0.013/mL | $0.65 | | |
| autosampler | 2 | $32/200 | $0.16/ea | $0.16 | | |
| vial | | | | | | |
| filter | 2 | $274.34/150 | $1.83/ea | $1.83 | | |
| syringe | 2 | $28.64/100 | $0.29/ea | $0.29 | | |
| scintilation | 2 | $128.55/500 | $0.26/ca | $0.26 | | |
| vial | | | | | | |
| deep well | 2 | $200/50 | $4/ea | $4.00 | | |
| well mat | 2 | $150/50 | $3/ea | $3.00 | | |
| column | 2 | $400/40 smp | $10/smp | $10.00 | $20.26 | $0.21 |
| column | 2 | $205/20 smp | $10.26/smp | $10.26 | | |
| guard | | | | | | |
| column | 2 | $460/ca | | | | |
| holder | | | | | | |

Total cost: $31.20/sample
$0.32/fracton

TABLE 6
Inhibition of COX-2 Peroxidase Activity by Extracts from Representative
Plant Species

| | | Inhibition |
|---|---|---|
| | Inhibition of COX-2 by | of COX-2 by |
| Plant Source | organic extract | aqueous extract |
| Scutellaria orthocalyx (root) | 55% | 77% |
| Scutellaria baicaensis (root) | 75% | 0% |
| Desmodium sambuense | 55% | 39% |
| (whole plant) | | |
| Eucaluptus globulus (leaf) | 30% | 10% |
| Murica nana (leaf) | 90% | 0% |

TABLE 7
IC₅₀ Values for Human and Ovine COX-2 and COX-1

| | IC₅₀ | IC₅₀ | IC₅₀ |
|---|---|---|---|
| Plant Source | Human COX-2 | Ovine COX-2 | Ovine COX-1 |
| Scutellaria | ND | 10 | 10 |
| orthocalyx (root) | | | |
| Scutellaria | 30 | 20 | 20 |
| baicalensis (root) | | | |
| Scutellaria lateriflora | 20 | 30 | 80 |
| (whole plant) | | | |
| Eucaluptus globulus | ND | 50 | 50 |
| (leaf) | | | |
| Murica nana | 5 | 6 | 7 |
| (leaf) | | | |

[image]

[image]

[image]

What is claimed is:

1. A method for discovering and developing novel therapeutic pharmaceutical, nutraceutical and cosmetic agents comprising the steps of:
(a) identifying and collecting a biological sample;
(b) extracting the sample using a two solvent system extraction procedure;
(c) separating the extracts using two separate high throughput (HTP) fractionating methods and simultaneously determining the activity of each HTP fraction;
(d) dereplicating the active fractions to identify the compounds present; and
(e) generating an indication, pharmacological and safety profile for each novel compound identified in step (d).

2. The method of claim 1 wherein the biological sample is selected from the group consisting of materials of botanic, microbial, fungal, mineral, marine, animal or human origin.

3. The method of claim 2 wherein said biological sample is a plant.

4. The method of claim 1 wherein the quantity of sample collected is from 1 gram to 10000 grams.

5. The method of claim 1 wherein said sample is selected based upon documented medicinal usage or mechanism of action.

6. The method of claim 1 further including the step of preparing a collection form for each sample collected.

7. The method of claim 6 wherein said collection form contains specific information about the sample including Latin name, distribution, collection location, therapeutic information, traditional preparations, botanical identification and published references.

8. The method of claim 7 wherein the information on said collection form is transferred to a database.

9. The method of claim 8 wherein the database is selected from the group of databases consisting of customerized Access, Oracle, Postgresql, Mysql and Sequl.

10. The method of claim 8 wherein the information in the database is stored in an individual table entered using an individual form.

11. The method of claim 8 further including the step of designing specific macros and queries to assess the of information and data stored in the database.

12. The method of claim 1 further including the step of preparing at least two specimen vouchers for each sample collected, wherein said specimen vouchers are comprised of dried, and/or preserved naturally and/or chemically the whole body of the sample including the full reproduction organs and wherein a taxonomy form is attached to each voucher specimen for purposes of identification.

13. The method of claim 1 wherein the solvent extraction procedure of step (b) further comprises the steps of:
(a) grinding an appropriate amount of sample;
(b) extracting the ground sample with a combination of two organic solvents, wherein said combination is comprised of a solvent of low polarity and a solvent of high polarity;
(c) drying the sample after organic extraction;
(d) extracting the dried sample with an aqueous solvent; and
(e) evaporating the solvent from both extractions and isolating the extract.

14. The method of claim 13 wherein the amount of sample is selected from 1 gram to 1000 grams.

15. The method of claim 13 wherein said low polarity is selected from the group consisting of an alkane having 6-10 carbons, a halogenated alkane having 1-4 carbon atoms, wherein each carbon atom has 1-4 halogen atoms, an ester having the formula R′COOR″, wherein R′ is selected from an alkyl group having between 1-6 carbons and R″ is selected from an alkyl group having between 1-8 carbons and a ketone having between 3-12 carbons.

16. The method of claim 13 wherein said low polarity solvent is selected from the group consisting of methylene chloride, ethyl acetate and chloroform.

17. The method of claim 13 wherein said high polarity solvent is selected from the group consisting of DMSO, THF and an alcohol wherein said alcohol has one to eight carbons.

18. The method of claim 17 wherein said alcohol is selected from the group consisting of methanol, ethanol, propanols and butanols.

19. The method of claim 13 wherein the quantity of solvents in both extractions is one to ten times the ratio of the weight of the extracted sample.

20. The method of claim 13 wherein the extraction is carried out by a method selected from the group consisting of shaking, sonication, refluxing, stirring, and pressurized mixing, and filtering.

21. The method of claim 1 wherein the extracts obtained from step (b) are prepared for bioassay by a method comprising the steps of
(a) weighing and dissolving the organic extract into a solvent;
(b) weighing and dissolving aqueous extract in a solvent; and
(c) transferring each extract solution into individual cell of a sample master plate.

22. The method of claim 21 wherein the solvent for dissolving the organic extract is selected from the group consisting of DMSO, DMF, THF, ketones having three to ten carbons and alcohols having one to five carbons.

23. The method of claim 21 wherein the solvent dissolving the aqueous extract is selected from the group consisting of water, DMSO, DMF, THF, ketones having three to ten carbons and alcohols having one to five carbons.

24. The method of claim 21 wherein the extract concentration in each solution is in the range of 0.0 mg to 1000 mg per milliliter solvent.

25. The method of claim 21 wherein the sample master plate is selected from the group consisting of a 96, 192, 384, 576, 768, 960, 1152, 1344 and 1536 well plate.

26. The method of claim 1 wherein the separation of the extracts comprises the steps of:
(a) using a parallel chromatography system or a high throughput purification (HTP) system;
(b) separating the organic extract with a normal phase pre-packed column;
(c) separating the aqueous extract with a reverse phase pre-packed column;
(d) detecting eluent with detector(s)
(e) collecting fractions; and
(f) evaporating the solvent.

27. The method of claim 26 wherein the chromatography system is comprised of two to four solvent delivery pumps, solvent mixers, and appropriate auto line switchers.

28. The method of claim 26 wherein the chromatography is carried out at ambient, low, medium or high solvent pressure.

29. The method of claim 26 wherein the chromatography is carried on at ambient, or a temperature from 20 to 80° C.

30. The method of claim 26 wherein the normal phase column is packed with a resin selected from the group consisting of silica gel, alumina, and amino propyl, cyano propyl, diol florisil or polyamide, ion exchange resins.

31. The method of claim 26 wherein the reverse phase column is packed with a resin selected from the group consisting of C-2, C-4, C-8, C-18, LH-20, XAD-4, XAD-16, and polystyrene-divinyl benzene based resins.

32. The method of claims 30 or 31 wherein the particle size of the resin in chromatography column is from 10 to 200 μm.

33. The method of claim 26 wherein the chromatography column is packed with 1 to 500 grams of resin.

34. The method of claim 26 wherein the normal phase chromatography column is eluted with a combination of three organic solvents selected from alkane having six to ten carbons, an organic ester, having the formula R¹COOR², wherein R¹ is selected from an alkyl group having between one to five carbon and R² is selected from an alkyl group having between one to six carbons, and an alcohol, having the formula R³OH, wherein R³ is an alkyl group having between one to six carbons.

35. The method of claim 26 wherein the reverse phase chromatography column is eluted with a combination of two solvents: DI water and a solvent selected from the group consisting of an alcohol with one to four carbons, acetonitrile, THF, or a ketone having three to twelve carbons.

36. The method of claim 26 wherein the detector is an ultraviolet (UV)/visual light detector comprising single or dual channels with single, continuing or broadband wavelength from 100-1000 nm.

37. The method of claim 26 wherein the detector is a MS detector comprising electronic spray ionization or sonic spray ionization chamber; ion trap or single or triple quadruple mass detection with positive or negative mode.

38. The method of claim 26 wherein the detector is a nuclear magnetic resonance (NMR) detector comprising a proton or a carbon probe.

39. The method of claim 26 wherein the detector is a reflex index (RI) detector.

40. The method of claim 26 wherein the detector is a light scattering detector (LSD).

41. The method of claim 26 further comprising the step preparing the extract fractions after step (d) for bioassay using a method comprising the steps of:
(a) dissolving the fractions from organic extract into a solvent;
(b) dissolving the fractions from aqueous extract into a solvent; and
(c) transferring the fraction solution into a sample plate

42. The method of claim 41 wherein the solvent for dissolving the fractions derived from the organic extract and the aqueous extract are independently selected from the group consisting of DMSO, DMF, THF, a ketone containing three to ten carbons, an alcohol containing one to five carbons and a combination of two to three of solvents.

43. The method of claim 41 wherein the extract concentration in the solution is between 0.001 mg to 100 mg/mL of solvent.

44. The method of claim 41 wherein the sample plate is selected from the group consisting of a 96, 192, 384, 576, 768, 960, 1152, 1344 and 1536 well plate.

45. The method of claim 1 wherein the dereplicating of the active fractions comprises the steps of:
(a) collecting activity data of the sample;
(b) collecting physical property, spectroscopic and structural data of the sample;
(c) analyzing the collected data;
(d) searching commercial databases for the properties of the sample; and
(e) reaching a conclusion regarding the active fractions.

46. The method of claim 45 wherein the activity measured is selected from the group consisting of enzyme inhibition, receptor binding, gene expression, cell function regulation, protein production, animal function regulation and animal physiological, neurological, and behavior function regulation, animal disease model manipulation and other measurements of biological function.

47. The method of claim 45 wherein the activity data is collected from extracts, fractions of extracts, purified compounds, semi-synthetic and synthetic compounds.

48. The method of claim 45 wherein the physical property data collected in the dereplication process is selected from retention time from a chromatogram based on absorption or changes of UV/VIS, refractive index, laser light scattering pattern, solvent elution volume, mass weight; pH, solubility and log P.

49. The method of claim 45 wherein the spectroscopic information collected is selected from UV/VIS spectrum, mass spectrum including molecular ion and fragmentation ions, NMR spectrum and light scattering spectrum.

50. The method of claim 45 wherein structural information is selected from mass fragmentation pattern and mass spectrum of daughter/grand daughter ions; chemical shifts of protons, carbons, phosphorous, and other elements from one and two dimensional nuclear magnetic resonance spectroscopic data; infrared spectrum and UV absorption spectrum.

51. The method of claim 45 wherein the physical property, spectroscopic and structure data is collected during separation of the extracts by splitting a fraction of eluent into one or more detectors.

52. The method of claim 45 wherein the physical property, spectroscopic and structure data are be collected by high pressure liquid chromatography (HPLC) from analysis of the individual fraction.

53. The method of claim 52 wherein the HPLC is comprised of two solvent pumps, a solvent mixer, a stainless steel column containing resin, a column oven and one or more detectors.

54. The method of claim 53 wherein the column is packed with a normal phase resin selected from the group consisting of silica gel, alumina, polyamide, amino propyl, cyano propyl, diol florisil and ion exchange resins.

55. The method of claim 53 wherein the column is packed with a reverse phase resin selected from the group consisting of a C-2, C-4, C-8, C-18, LH-20, XAD-4, XAD-16 and polystyrene-divinyl benzene based polymer.

56. The method of claim 53 wherein the particle size of the resin is selected from 1 to 100 μm.

57. The method of claim 53 wherein the chromatography column contains from 0.1 to 50 grams of resin.

58. The method of claim 45 wherein the commercial databases are selected from the group consisting of the Dictionary of Natural Products, Chemical Abstracts Service's Registration File, NAPROLERT, MEDLINE, NERAC, DEREP and the Bioactive Natural Product Database.

59. The method of claim 1 wherein the novel ingredient is identified by a bioassay directed isolation, purification and identification process.

60. The method of claim 1 wherein the pharmacology profile is the ability to modulate the activity and function of a biological system, biochemical materials, and gene targets.

61. The method of claim 60 wherein the ability to modulate the activity and function is determined from measurement of biological functions selected from the group consisting of enzyme inhibition, receptor binding, gene expression, cell function regulation, protein production, animal function regulation and animal disease model manipulation.

62. The method of claim 61 wherein the gene target is the expression of a disease or metabolism, or physiology related gene.

63. The method of claim 62 wherein the gene or a portion thereof is of human origin.

64. The method of claim 62, wherein the disease-related gene is associated with a disease selected from the group consisting of cardiovascular disease, respiratory disease, disease of the kidney, disease of the liver, disease of the pancreas, gastrointestinal disease, hematological disease, metabolic disease, neurological disease, aging, immune disease, disease of the reproductive system, infectious disease and skeletal disease.

65. The method of claim 62 wherein the disease-related gene is associated with a conditions selected from the group consisting of inflammation, the immune response, energy metabolism, wound healing, allergy, menopause, aging, oxidative stress and cancer.

66. The method of claim 62 wherein the expression of the disease-, metabolism- or physiology-related gene is measured by the level of messenger RNA of such gene.

67. The method of claims 62 wherein the expression of the of the disease-, metabolism- or physiology-related gene is measured by a method selected from the group consisting of Northern blot analysis, dot blot hybridization, DNA microarray hybridization and quantitative polymerase chain reaction (gPCR).

68. A method of claim 1 wherein the safety profile is determined by measurement of the ability to maintain the normal activity and function of the biological system, biochemical materials, and molecular biology targets while administrating considerable amount of the compound.

69. The method of claim 1 further comprising the step of developing the novel compound identified into a commercially viable product.

70. The method of claim 13 wherein said an aqueous solvent is selected from the group consisting of water, acidic water, basic water and buffer solutions.

71. The method of claim 70 wherein said acid, basic and buffer solutions are selected from organic or inorganic acid, base, and salts at a pH range from one to fourteen.
